(19) United States
(12) Patent Application Publication
(10) Pub. No.: US 2025/0276307 A1
(43) Pub. Date: Sep. 4, 2025

(54) CATALYST COMPOSITIONS AND METHODS OF PREPARATION AND USE THEREOF

(71) Applicant: BASF CORPORATION, FLORHAM PARK, NJ (US)

(72) Inventors: Jian-Ping Chen, Hudson, OH (US); Arunabha Kundu, Richmond Heights, OH (US); Scott Hedrick, Solon, OH (US)

(73) Assignee: BASF CORPORATION, FLORHAM PARK, NJ (US)

(21) Appl. No.: 18/858,507

(22) PCT Filed: Apr. 18, 2023
(86) PCT No.: PCT/US2023/018914

§ 371 Date: Oct. 21, 2024

Related U.S. Application Data
(60) Provisional application No. 63/334,414, filed on Apr. 25, 2022.

(51) Int. Cl. B01J 23/83; B01J 21/04; B01J 35/30; B01J 35/32; B01J 35/40; B01J 35/54; B01J 35/61; B01J 35/63; B01J 35/64; B01J 37/00; B01J 37/03; B01J 37/08; C07C 29/149

(52) U.S. Cl. CPC B01J 23/83; B01J 21/04; B01J 35/32; B01J 35/393; B01J 35/394; B01J 35/40; B01J 35/54; B01J 35/613; B01J 35/615; B01J 35/633; B01J 35/647; B01J 37/009; B01J 37/035; B01J 37/08; C07C 29/149

(57) ABSTRACT

Disclosed herein are chromium-free catalyst compositions having an alumina support and a copper compound on the alumina support. The catalyst composition may further include a promoter. Further disclosed are methods of preparing such catalyst compositions and methods of use thereof.

### CLAIM OF PRIORITY

The present application claims the benefit under 35 U.S.C. § 119(e) of U.S. Provisional Patent Application No. 63/334,414 filed Apr. 25, 2022, which is incorporated by reference herein.

### FIELD

Embodiments of the present disclosure relate to chromium-free copper catalyst compositions and methods of preparation and use thereof.

### BACKGROUND

Some current commercial manufacturing processes for ester or carbonyl compound hydrogenation/hydrogenolysis to their corresponding alcohol use chrome-containing copper catalysts. Chrome-containing catalysts are considered hazardous chemicals that impact human health and pollute the environment under Registration, Evaluation, Authorization and Restriction of Chemicals (REACH) regulations. In accordance with such REACH regulations, the substance Chromium (VI) trioxide (CrO₃) shall not be placed on the market for use nor used in the European Union. Chrome-containing catalysts can potentially contain trace Cr (6+) as impurity.

### BRIEF SUMMARY

According to various embodiments, disclosed herein are catalyst compositions. In some embodiments, the catalyst compositions comprise a support comprising alumina, a copper compound on the support, and a promoter, wherein the catalyst composition is free of chromium.

Further disclosed herein are methods of preparing a catalyst composition. In some embodiments, the methods include combining a copper containing solution with alumina to form a combination, precipitating the combination with a base solution to form a precipitate solution, filtering the precipitate solution to obtain a precipitate, and calcining the precipitate to form the catalyst composition.

In further embodiments, disclosed herein are methods for the hydrogenation or hydrogenolysis of organic compounds comprising carbonyl groups. According to embodiments, the methods can include contacting the organic compounds with a catalyst composition according to embodiments herein, wherein the catalyst composition is free of chromium.

### BRIEF SUMMARY OF THE DRAWINGS

The present disclosure is illustrated by way of example, and not by way of limitation, in the figures of the accompanying drawings in which like references indicate similar elements.

FIG. 1 is a chart showing the incremental pore volume as a function of pore diameter for exemplary catalysts calcined at 500° C. (Examples 6 and 8) and 750° C. (Examples 7-3 and 9) in accordance with embodiments herein prepared in Examples 6-9.

FIG. 2 is a chart showing the cumulative pore area as a function of pore diameter for exemplary catalysts calcined at 500° C. (Examples 6 and 8) and 750° C. (Examples 7-3 and 9) in accordance with embodiments herein prepared in Examples 6-9.

FIG. 3 is a chart showing the incremental pore volume as a function of pore diameter for exemplary catalysts calcined at 500° C. (Examples 6 and 8) and 750° C. (Examples 7-3 and 9) in accordance with embodiments herein prepared in Examples 6-9.

FIG. 4 is a chart showing the cumulative pore area as a function of pore diameter for exemplary catalysts calcined at 500° C. (Examples 6 and 8) and 750° C. (Examples 7-3 and 9) in accordance with embodiments herein prepared in Examples 6-9.

FIG. 5 is a chart showing the x-ray diffraction (XRD) patterns for catalysts calcined at 500° C. (Examples 6 and 8) and 750° C. (Examples 7-3 and 9) in accordance with embodiments herein prepared in Examples 6-9.

FIG. 6 is a chart showing the XRD patterns for catalysts prepared using dry powder from Example 6, calcined at different temperatures, in accordance with embodiments herein prepared for Examples 6 and 7-1 to 7-4.

FIG. 7 is a chart showing the copper oxide crystallite size as a function of calcination temperature for a catalyst according to embodiments herein prepared in Example 7.

FIG. 8 is a chart showing the performance in terms of fatty alcohol yield of unpromoted Cu—Al₂O₃ catalyst (Example 1) in comparison to a CuCr reference Example 1.

FIG. 9 is a chart showing the performance in terms of fatty alcohol yield of Mn promoted Cu—Al₂O₃ catalyst (Example 2) in comparison to CuCr reference Example 1.

FIG. 10 is a chart showing the performance in terms of fatty alcohol yield of Mn promoted Cu—Al₂O₃ catalyst calcined at 500° C. and 750° C. respectively (Examples 3 and 4) in comparison to CuCr reference Example 1.

FIG. 11 is a chart showing the performance in terms of fatty alcohol yield of La promoted Cu—Al₂O₃ catalyst (Examples 5, 6 and 8) in comparison to CuCr reference Example 1.

FIG. 12 is a chart showing the performance in terms of fatty alcohol yield of La promoted Cu—Al₂O₃ catalyst calcined at 500° C. and 750° C. respectively (Examples 6 and 7-3) in comparison to CuCr reference Example 1.

FIG. 13 is a chart showing a multi-cycle performance test for a CuCr reference catalyst composition (Example 1) according to embodiments herein.

FIG. 14 is a chart showing a multi-cycle performance test for a catalyst composition (Example 6) according to embodiments herein.

FIG. 15A a chart showing a comparison between a CuCr reference catalyst (Example 1) and the catalyst compositions of Examples 3 and 6 for the first cycle (fresh) of a multi-cycle performance test.

FIG. 15B a chart showing a comparison between a CuCr reference catalyst (Example 1) and the catalyst compositions of Examples 3 and 6 for the second cycle (first re-use) of a multi-cycle performance test.

FIG. 15C a chart showing a comparison of fatty alcohol yield between a CuCr reference catalyst (Example 1) and the catalyst compositions of Examples 3 and 6 for the third cycle (second re-use) of a multi-cycle performance test.

FIG. 15D a chart showing a comparison of fatty alcohol yield between a CuCr reference catalyst (Example 1) and the catalyst compositions of Examples 3 and 6 for the fourth cycle (third re-use) of a multi-cycle performance test.

FIG. 15E a chart showing a comparison of fatty alcohol yield between a CuCr reference catalyst (Example 1) and the catalyst compositions of Examples 3 and 6 for the fifth cycle (fourth re-use) of a multi-cycle performance test.

FIG. 16 is a chart showing a comparison between a CuCr reference catalyst (Example 1) and the catalyst compositions of Example 6 for each cycle of re-use in the hydrogenolysis of methyl ester to form fatty alcohol.

### DETAILED DESCRIPTION

In view of the pollution and health hazards, it is desirable to replace chromium-containing copper catalysts with chromium-free catalysts. As such, disclosed herein are embodiments of a chromium-free copper catalyst. Chromium-free promoted copper catalysts according to various embodiments herein can be used to replace the currently used copper-chrome (CuCr) catalysts that pose such environmental and health issues.

### Catalyst Compositions

Catalyst compositions according to embodiments herein contain a support (e.g., comprised of alumina, pseudo boehmite, gamma alumina, VERSAL™ V-250 alumina powder). The support is configured to support a metal (e.g., copper) and/or other promoters such as La, Mn, Ba, Zr and Mg, etc. Supports formed of alumina powder can form a stable catalyst with a desirable powder particle size distribution for the final catalyst. A powder catalyst composition with appropriate particle size distribution leads to improved liquid product separation by filtration or centrifuge and to a more stable catalyst in slurry phase processes. Such supported powder catalyst compositions having an alumina powder support results in providing more resistance to free organic acid present in the feed than co-precipitated catalysts. In various embodiments, the catalyst compositions contain copper and are chromium-free. The catalyst compositions according to one or more embodiments also may be in any suitable form including, but not limited to, tablets and extrudates.

The catalyst compositions can include one or more promoter. Adding promoters to alumina supported catalysts can improve further catalyst activity and stability by additional interaction of the promoter to the active metal and support. Suitable promoters include, but are not limited to, manganese (Mn), lanthanum (La), barium (Ba), calcium (Ca), magnesium (Mg), zirconium (Zr), strontium (Sr) and combinations thereof. Powder catalysts with one or more promoters can exhibit higher product yield and more stability toward impurities poisoning, such as, free acid, etc. Promoted Cu on alumina catalysts can have stable catalytic activity after multiple reuses in a slurry phase hydrogenation process. In some embodiments, promoted catalysts according to embodiments herein have higher resistance toward chemical attacks, such as feedstock containing organic acids which will lead to prolonged catalyst life.

According to at least one embodiment, formed catalysts, such as tablets, exhibit advantageous physical properties and stability with promoters such as Mn and La. With these promoters, shrinkage upon higher temperature calcination is minimized. Such catalysts are particularly useful for hydrogenating organic compounds containing carbonyl groups such as aldehydes, esters, ketones and carboxylic acids.

In some embodiments, the catalyst compositions include about 35 wt % to about 70 wt % CuO, about 0 wt % to about 12 wt % BaO, about 0 wt % to about 12 wt % La₂O₃, about 0 wt % to about 15 wt % Mn₂0₃ and about 20 wt % to about 40 wt % Al₂O₃. The catalyst can have crystal phases of monoclinic copper (II) oxide (CuO), and one or more of the following crystal phases: monoclinic lanthanum oxide carbonate (LaCO₃), orthorhombic lanthanum copper oxide (La₂CuO₄), tetragonal copper lanthanum oxide (CuLaO₃), copper aluminum oxide (CuAl₄O₇), cubic copper aluminum oxide (CuAl₂O₄), monoclinic lanthanum manganese oxide (LaMnO_{3.13}), cubic copper manganese oxide (Cu_{1.5}Mn_{1.5}O₄), orthorhombic lanthanum copper oxide (La₂CuO₄), cubic aluminum oxide (Al₂O₃) or alumina, etc. The BET SA can be about 80 m²/g to about 160 m²/g, and the pore volume can be about 0.25 ml/g to about 0.4 ml/g.

In embodiments, where the catalyst composition is in the form of subunits such as particles, granules, powder, spheres, extrudates, micro-tablets, etc., subunits can have an average particle size distribution as follows: D₁₀ about 1 micron to about 3 microns, D₅₀ about 10 microns to about 20 microns and D₉₀ 30 microns to about 40 microns. The loose packed bulk density can be about 0.25 g/ml to about 0.6 g/ml and the CuO crystallite size of about 90 Å to 220 Å.

When the catalyst composition is in a solid form, such as ⅛″ tablet, the catalyst solid can have a crush strength of about 10 lbs to about 40 lbs. The bulk density can be about 1.0 g/ml to about 1.7 g/ml, the BET SA about 20 m²/g to about 100 m²/g, a pore volume of about 0.25 ml/g to about 0.4 ml/g and an average pore diameter (as measured and calculated by 4V/A) of about 80 Å to about 160 Å.

With addition of promoters (such as La and Ba), the catalyst compositions can have better stability to chemical attack, such as multiple re-uses when the feed contains an organic acid. Also, catalyst compositions according to embodiments herein can have stronger mechanical strength especially after heat treatment at a higher temperature and no or minimum shrinkage with a La promoter. Mn as a promoter also increases catalyst hydrogenation and hydrogenolysis activity and selectivity toward alcohol product.

According to various embodiments, catalyst compositions disclosed herein can include a support. The support can be comprised of any suitable material. Suitable materials include, but are not limited to, alumina, pseudoboehmite alumina, gamma alumina, VERSAL™ V-250alumina powder, bayerite alumina, or combinations thereof. The catalyst composition can include the support material in an amount of about 10 wt % to about 50 wt %, about 15 wt % to about 45 wt %, or about 20 wt % to about 40 wt %, based on the total weight of the catalyst composition.

According to various embodiments, the support has a bulk density of about 1 lbs/ft³ to about 60 lbs/ft³, about 5 lbs/ft³ to about 50 lbs/ft³, or about 10 lbs/ft³ to about 40 lbs/ft³, or any individual value or sub-range within these ranges. In some embodiments, the support has a bulk density of about 1 lbs/ft³ to about 60 lbs/ft³, about 5 lbs/ft³ to about 50 lbs/ft³, or about 10 lbs/ft³to about 40 lbs/ft³, or any individual value or sub-range within these ranges. According to some embodiments, the support has a surface area of about 250 m²/g to about 400 m²/g, about 275 m²/g to about 375 m²/g, or about 300 m²/g to about 350 m²/g.

In some embodiments, one or more metal or metal-containing compound may be adsorbed onto the support. Suitable metals or metal-containing compounds include, but are not limited to copper, copper oxides, copper-containing compounds, copper (I) oxide (cuprous oxide), copper (II) oxide (cupric oxide) or combinations thereof. The copper compound may be copper (II) oxide comprising a crystallite size of about 90 Å to about 200 Å. In one or more embodiments, the catalyst composition can include monoclinic copper (II) oxide (CuO), and can further include one or more of the following crystal phases: monoclinic lanthanum oxide carbonate (LaCO₃); orthorhombic lanthanum copper oxide (La₂CuO₄); tetragonal copper lanthanum oxide (CuLaO₃); copper aluminum oxide (CuAl₄O₇); cubic copper aluminum oxide (CuAl₂O₄); monoclinic lanthanum manganese oxide (LaMnO_{3.13}); cubic copper manganese oxide (Cu_{1.5}Mn_{1.5}O₄); orthorhombic lanthanum copper oxide (La₂CuO₄); and/or cubic aluminum oxide (Al₂O₃).

In some embodiments, the catalyst composition is free of one or more of chromium, aluminum nitrate, an acetate, a chloride or sodium aluminate. According to various embodiments, a catalyst composition according to embodiments herein can include the copper compound in an amount, based on the total weight of the catalyst composition, of about 25 wt % to about 80 wt %, about 35 wt % to about 70 wt %, or any individual value or sub-range within these range.

In at least one embodiment, the catalyst composition can include a promoter. Suitable promoters include, but are not limited to, lanthanum, manganese, barium, zirconium, calcium, magnesium, zinc, yttrium oxide, erbium oxide, cerium oxide, a rare earth oxide, strontium, boron, nickel, platinum, silver, gold, palladium, ruthenium or combinations thereof. In some embodiments, the catalyst composition contains one or more promoter in an amount of about 0 wt % to about 20 wt %, about 1 wt % to about 15 wt %, or about 5 wt % to about 12 wt %, or any individual value or sub-range within these ranges. In some embodiments, the promoter comprises barium oxide in an amount of about 0 wt % to about 20 wt %, or about 1 wt % to about 15 wt %, or about 5 wt % to about 12 wt %, based on the total weight of the catalyst composition. In one or more embodiments, the promoter comprises lanthanum oxide (La₂O₃) in an amount of about 0 wt % to about 20 wt %, or about 1 wt % to about 17 wt %, or about 5 wt % to about 15 wt %, based on the total weight of the catalyst composition. The promoter may include manganese oxide (Mn₂O₃) in an amount of about 0 wt % to about 20 wt %, or about 1 wt % to about 17 wt %, or about 5 wt % to about 15 wt %, based on the total weight of the catalyst composition.

In one or more embodiments, the catalyst composition is in the form of a powder, granules, extrudates, spheres, a solid, tablet, caplet, slug, or combinations thereof. According to at least one embodiment, the catalyst composition is a powder comprising an average particle size of D₁₀ of about 1 μm to about 10 μm, D₅₀ of about 10 um to about 20 μm and D₉₀ of about 30 μm to about 60 μm. In some embodiments, the catalyst composition is in powder form and comprises a loose packed bulk density of about 0.25 g/ml to about 0.6 g/m, or wherein the catalyst is in tablet form and comprises a bulk density of greater than about 1.0 g/ml.

According to one or more embodiments, the catalyst composition has a stability against impurity poisoning of about 0.1% to about 0.5% of organic acid or up to about 0.5% of organic acid. In some embodiments, the catalyst composition has a stability when in a slurry phase of about 1 re-use to about 10 re-uses, about 1 re-use to about 5 re-uses, about 1 re-use to about 4 re-uses, about 2 re-uses to about 5 re-uses when measured using a multiple-cycle performance test for fatty alcohol production. In at least one embodiment, the catalyst composition has a fatty acid conversion of about 73% to about 99%, or at least about 73% based on the % saponification (SAP) value reduction.

The catalyst composition may be calcined. A calcined catalyst composition contains less moisture and/or impurities than a non-calcined catalyst composition. In some embodiments, the size of the catalyst composition pre-calcination is the same or about the same as the size of the catalyst composition post-calcination.

Catalyst compositions as described herein can have a BET surface area of about 60 m²/g to about 200 m²/g, about 70 m²/g to about 180 m²/g, or about 80 m²/g to about 160 m²/g, or any individual value or sub-range within these ranges. In some embodiments, the catalyst composition is in the form of at least one of a sphere, solid, tablet, caplet or slug having a thickness of about ¼ in to about 1/16 in, or about ⅛ in, or any individual thickness or sub-range within this range. The catalyst composition may be in any of the aforementioned forms and can have a crush strength of about 5 lbs to about 40 lbs, or any individual value or sub-range within this range. In some embodiments, the catalyst composition can have a bulk density of about 1.0 g/ml to about 1.7 g/ml, or any individual value or sub-range within this range. In one or more embodiments, catalyst compositions as described herein can have a BET surface area (SA) of about 20 m²/g to about 100 m²/g, or any individual value or sub-range within this range. Catalyst compositions as described herein can include a pore volume of about 0.25 ml/g to about 0.4 ml/g, or any individual value or sub-range within this range. In some embodiment, catalyst compositions as described herein can have an average pore diameter of about 80 Å to about 160 Å as measured and calculated by 4 volume/area (V/A), or any individual value or sub-range within this range.

### Methods of Preparing Chromium-Free Catalyst Compositions

Catalyst compositions according to embodiments herein can be prepared using deposition-precipitation of a metal or metal-containing compound (e.g., CuO) and one or more promoters (e.g., oxide promoters) on a support material (e.g., alumina powder). The resulting catalyst compositions can have improved stability and higher activity and selectivity for hydrogenation and hydrogenolysis of carbonyl-containing organic compounds as compared to conventional catalyst compositions. Previous alumina-containing copper catalysts were prepared by co-precipitation using alumina salts, such as aluminum nitrate, acetate, chlorides or sodium aluminate as alumina raw material and such compositions did not show the same levels of stability, activity or selectivity for hydrogenation and hydrogenolysis of carbonyl-containing organic compounds as compared to catalyst compositions according to embodiments herein.

Further described herein are methods of preparing chromium-free catalyst compositions. In some embodiments, the catalyst composition is formed by deposition-precipitation where alumina powder is combined with an initial water heel and the other metal forming a slurry that is deposited onto a support (e.g., alumina powder).

In one or more embodiments, a method of preparing a catalyst composition includes combining a copper containing solution with alumina to form a combination. The combination can be a slurry, a suspension, a dispersion, a liquid, a mixture or any combination thereof. The copper containing solution can include, but is not limited to copper (II) nitrate (Cu(NO₃)₂), copper (I) chloride, copper (II) acetate or combinations thereof. The alumina can be in the form of a powder, granules, extrudates, spheres, a solid, tablet, caplet, slug, or combinations thereof. In some embodiments, the alumina can include pseudoboehmite alumina, bayerite alumina, gamma alumina or combinations thereof.

The method can further include precipitating the combination with a base solution to form a precipitate solution. In some embodiments, precipitating the combination comprises combining the combination with a base. The base can include, but is not limited to, sodium hydroxide (NaOH), soda ash, or a combination thereof. According to embodiments, the combination is maintained at a pH of about 6.5 to about 7.8 while precipitating the combination.

The method can further include filtering the precipitate solution to obtain a precipitate. The precipitate is the solid material that is collected by the filter. The precipitate can be in the form of sub-units such as particles, agglomerates, flakes, etc. In some embodiments, the method includes washing the precipitate and subsequently drying the washed precipitate.

In one or more embodiments, the method further includes calcining the precipitate to form the catalyst composition. Calcining the precipitate can be at a temperature of about 200° C. to about 800° C., 300° C. to about 700° C., about 400° C. to about 600° C., or about 450° C. to about 500° C. for about 30 minutes to about 4 hours, about 1 hour to about 3 hours, about 1.5 hours to about 2.0 hours. The resulting catalyst compositions may be free of chromium. In some embodiments, the catalyst composition is also free of aluminum nitrate, an acetate, a chloride or sodium aluminate.

In some embodiments, the prepared catalyst composition can include about 40 wt % to about 70 wt %, or about 55 wt % to about 65 wt % copper (II) oxide (CuO) and about 30 wt % to about 60 wt %, or about 40 wt % to about 50 wt % alumina. The catalyst composition can have a loose packed bulk density of about 0.1 g/cc to about 0.5 g/cc, or about 0.2 to about 0.3 g/cc. In some embodiments, the catalyst composition can have a packed bulk density of about 0.2 g/cc to about 0.6 g/cc, or about 0.3 to about 0.4 g/cc. In some embodiments, the catalyst composition is a powder having an average particle size of D₁₀ of about 1 μm to about 10 μm, D50 of about 10 μm to about 20 μm and D90 of about 30 μm to about 60 μm.

### Methods of Using Chromium-Free Catalyst Compositions

In some embodiments, catalyst compositions according to embodiments herein (e.g., promoted CuO on alumina powder catalysts) may be used for hydrogenation or hydrogenolysis of carbonyl group containing compounds, especially methyl esters, dimethyl esters, wax esters, aldehydes, ketones, and carboxylic acids. Methods for the hydrogenation or hydrogenolysis of organic compounds comprising carbonyl groups can include contacting the organic compounds with a catalyst composition according to embodiments herein. The catalyst composition can be free of chromium.

In some embodiments, the organic compounds comprise one or more of aldehydes, esters, ketones or carboxylic acids. The organic compounds can include one or more of fatty esters or methyl ester. In some embodiments, the organic compounds comprise a methyl ester that undergoes hydrogenolysis to form one or more fatty alcohols. In one or more embodiments, the organic compounds comprise a wax ester that undergoes hydrogenolysis to form one or more fatty alcohols.

### Examples

These catalyst samples were characterized and evaluated for catalytic performance for fatty ester hydrogenolysis applications. The details of the catalyst preparation procedure are illustrated by the following examples.

### Reference Example 1 CuCr Catalyst

A commercial copper chrome catalyst was obtained for.

### Reference Example 2 Cu—Al₂O₃ Catalyst

A Cu—Al₂O₃ catalyst was prepared using a co-precipitation method. The Cu—Al₂O₃catalyst was prepared by weighing out 1640 g of a copper nitrate solution (15.48% Cu), which was subsequently diluted with deionized water to 2500 ml. The preparation method further included weighing out 815.6 g of a sodium aluminate (25% Al₂O₃) and diluting the solution with deionized water to 2500 ml.

A 12-liter tank was filled with 2500 ml of deionized water. The method subsequently weighed out 318 g sodium carbonate powder, which was dissolved in deionized water to 1500 ml. Simultaneously the copper nitrate solution and sodium aluminate solution were added to the 2500 ml of deionized water. The copper nitrate and sodium aluminate solutions were added at a rate of 33 ml per minute. A sodium carbonate (soda ash) solution was added to the mixture, keeping the slurry at a constant pH around 7.4, by adjusting the rate of addition of the soda ash solution. Precipitation was carried out at room temperature. The resulting slurry was filtered to form a filter cake. The filter cake was washed with 3000 ml of deionized water three or more times. The filter cake was dried at 120° C. overnight. The dried CuAl powder was calcined at 700-800° C. for 2 hours.

### Reference Example 3 Cu—Mn—Al₂O₃

A Cu—Mn—Al₂O₃ catalyst Cu was prepared using a co-precipitation method. A Cu(NO₃)₂ solution was weighed out to 984.6 g. Added to this Cu(NO₃)₂ solution was 200.3 g of a Mn(NO₃)₂ solution. The resulting mixture was diluted to IL with deionized water. The preparation method included weighing 439 g of Na₂Al₂O₄, which was dilute to 667 mL with deionized water. A 1.33 L deionized water heel was placed in a baffled strike tank with an agitation rate at 400 RPM. An amount of 267 g of Na₂CO₃ was weighed out and diluted to 1.33 L with deionized water. The CuMn solution was added at 16 mL/min simultaneously with the Al solution at 11 mL/min. The sodium carbonate (soda ash) solution was added to the mixture, keeping the slurry at a constant pH of around 7.0, by adjusting the rate of addition of the soda ash solution at room temperature. The slurry was filtered to collect a filter cake solid. The filter cake was washed with deionized water and dried at 120° C. overnight. The dry powder was calcined at 750° C. for 4 hours. The resulting preparation had a chemical composition 58% CuO-12% Mn₂O₃-30% Al₂O₃.

These reference catalysts were used to illustrate the superior performance of the inventive catalysts when applied to fatty ester hydrogenolysis. Thus, an improvement over the prior art Cu—Al₂O₃ and Cu—Mn—Al₂O₃ catalysts was demonstrated.

### Inventive Examples

Catalyst compositions according to various embodiments described herein were prepared. The following list of raw materials were used to prepare the inventive examples.
UOP V-250 alumina powder
NaOH 50% solution
Na₂CO₃ Sodium Carbonate
Cu(NO₃)₂ Copper Nitrate 17%
La(NO₃)₃
Mn(NO₃)₂
BaCO₃
Ba(NO₃)₂

### Example 1—Chromium-Free Copper Catalyst Composition

The following laboratory procedure was used to prepare a CuO on Al₂O₃ catalyst composition according to embodiments described herein:
1) Weigh out 2100 g of Cu(NO₃)₂ Soln. (16.16% Cu, from plant), dilute with D.I. to 2 liters.
2) DI water heel of 2 liters and set mixing speed at 400 RPM.
3) Weigh out 287 g of V-250 UOP alumina powder and slowly add into heel. Keep agitation until alumina powder is totally dispersed in the slurry.
4) Weigh out 250 g of 50% NaOH, dissolve in D.I. dilute to 2.75 liters. Weigh the solution before and after precipitation to get the amount of soda ash consumed.
5) Keep the slurry pH=7.0 by adjusting base solution addition rate (room temperature)
6) Precipitation is completed by adding copper nitrate and base solution simultaneously in one hour
7) Measure slurry particle size throughout the precipitation
8) Filter the slurry and wash the filter cake
9) Dry the washed filter cake to form powder
10) Calcine the dried powder at 500° C. for 2 hours to form final catalyst powder

The resulting calcined powder catalyst contained about 60% CuO, the balance being alumina. Other properties of the catalyst included a Loose Apparent Bulk Density (ABD) of 0.27 g/cc and a Packed ABD of 0.37 g/cc. The catalyst had the following particle size distribution: D₁₀=6.2 microns, D₅₀=16.7 microns and D₉₀=56.5 microns.

### Example 2—Chromium-free Copper Catalyst Targeted Composition—58% CuO-12% M₂O₃ 30%-Al₂O₃

The following laboratory procedure was used to prepare Cu—Mn—Al₂O₃ (58% CuO-12% MnOX 30%-Al₂O₃), 34846-43-2 and 34846-47-3 catalyst compositions according to embodiments described herein:
1) Weigh out 1400 g of Cu(NO₃)₂ Soln. (16.16% Cu, from plant),
2) Weigh out 311 g of manganese nitrate solution
3) Mixed solution 1 and 2 and dilute with deionized (DI) water to 1.33 Liters.
4) Water heel of 2 liters and set mixing speed at 400 RPM.
5) Weigh out 191 g V-250 UOP alumina powder and slowly add into heel. Keep agitation until alumina powder is totally dispersed in the slurry.
6) Weigh out 750 g soda ash, dissolve in 2.75 liters of DI water. Weigh the solution before and after precipitation to get the amount of soda ash consumed.
7) Keep the slurry pH=7.0 by adjusting base solution addition rate (room temperature)
8) Precipitation completed by adding copper nitrate and base solution simultaneously in one hour
9) Measure slurry particle size throughout the precipitation
10) Filter the slurry and wash the filter cake
11) Dry the washed filter cake to form powder
12) Calcine that dry powder at 500° C. for 2 hours

The resulting dry powder had the following main properties: Particle size distribution of this catalyst was D₁₀, 3.3 microns, D₅₀, 12.7 microns, D₉₀, 126.9 microns; Loose Apparent Bulk Density (LABD)=0.37 g/ml; and Packed Apparent Bulk Density (PABD)=0.47 g/ml.

### Example 3—Chromium-free Copper Catalyst Targeted Composition—61% CuO-6.5% Mn₂O₃-30% Al₂O₃

The following laboratory procedure was used to prepare a 61% CuO-6.5% Mn₂O₃-30% Al₂O₃ catalyst composition according to embodiments described herein:
1) Weigh out 1400 g of Cu(NO₃)₂ solution (16.16% Cu, from plant),
2) Weigh out 156 g of manganese nitrate solution
3) Mixed solution 1 and 2 and dilute with DI water to 1.33 Liters
4) Water heel of 2 liters and set mixing speed at 400 RPM
5) Weigh out 191 g of V-250 UOP alumina powder and slowly add into heel. Keep agitation until alumina powder is totally dispersed in the slurry
6) Weigh out 750 g of soda ash, dissolve in 2.75 liters of DI water. Weigh the solution before and after precipitation to get the amount of soda ash consumed
7) Keep the slurry pH=7.0 by adjusting base solution addition rate (room temperature)
8) Precipitation completed by adding copper nitrate and base solution simultaneously in one hour
9) Measure slurry particle size throughout the precipitation
10) Filter the slurry and wash the filter cake
11) Dry the washed filter cake to form powder
12) Calcine that dry powder at 500° C. for 2 hours

The dry powder had the following chemical compositions: 61.5% CuO, 6.3% Mn₂O₃ and 30.1% Al₂O₃. The particle size distribution of this catalyst was: D₁₀, 4.3 microns, D₅₀, 29.0 microns and D₉₀, 128.5 microns. The final calcined catalyst had the following properties: LABD−0.28 g/ml, PABD=0.36 g/ml and BET=127 m²/g.

Tablets were prepared from the dry powder. The tablets had a size of 3 mm×3 mm tablets and were made by adding 2-3% graphite into the powder, which was calcined at 500° C. The tablet was finally calcined at 750° C. for 4 h. The tablet properties were as follows:
Side crush strength (lbs): 45
BET surface area (m²/g): 48
Average bed density (g/mL): 1.5
N₂ pore volume (cm³/g): 0.14
Mean pore diameter (nm): 128.2
Cu dispersion (%): 1.7

### Example 4—Chromium-Free Copper Catalyst Composition

Procedures for the precipitation and drying were the same as in Example 3. The final calcination was at 750° C. for 2 hours. The resulting dry powder had the same chemical compositions as Example 3:61.5% CuO, 6.3% Mn₂O₃ and 30.1% Al₂O₃. The particle size distribution of the catalyst was: D₁₀, 4.3 microns, D₅₀, 29.0 microns and D₉₀, 128.5 microns.

### Example 5-Chromium—Free Copper Catalyst Composition—Cu—La—Al₂O₃ (Low La)

The following laboratory procedure was used to prepare a Cu—La—Al₂O₃ (low La). The catalyst composition according to embodiments described herein:
1) Weigh out 1400 g of Cu(NO₃)₂ Soln. (16.16% Cu, from plant),
2) Weigh out 53 g of lanthanum nitrate solution (22% La solution)
3) Mix solution 1 and 2 and dilute with DI water to 1.33 Liters.
4) Water heel of 2 liters and set mixing speed at 400 RPM.
5) Weigh out 191 g of V-250 UOP alumina powder and slowly add into heel. Keep agitation until alumina powder is totally dispersed in the slurry.
6) Weigh 750 g of soda ash, dissolve in 2.75 liters of DI water. Weigh out the solution before and after precipitation to get the amount of soda ash consumed.
7) Keep the slurry pH=7.0 by adjusting base solution addition rate (room temperature)
8) Precipitation completed by adding copper nitrate and base solution simultaneously in one hour
9) Measure slurry particle size throughout the precipitation
10) Filter the slurry and wash the filter cake
11) Dry the washed filter cake to form powder
12) Calcine the dry powder at 500° C. for 2 hours

Analytical results showed that this catalyst has the following composition: 66.3% CuO-3.2% La₂O₃-30.5% Al₂O₃.

Tablets were prepared from the dry powder. The tablets had a size of 3 mm×3 mm and were made by adding 2-3% graphite into the dry powder, which was calcined at 500° C. The tablet was finally calcined at 750° C. for 4 h. The resulting tablet had the following properties: Side crush strength (lbs): 29, BET surface area (m²/g): 52, Average bed density (g/mL): 1.42 and N2 pore volume (cm³/g): 0.16.

### Example 6—Chromium-Free Copper Catalyst Composition—Cu—La—Al₂O₃ (Mid La)

The following laboratory procedure was used to prepare a Cu—La—Al₂O₃ (mid La). The catalyst composition according to embodiments described herein:
1) Weigh out 1400 g of Cu(NO₃)₂ soln. (16.16% Cu, from plant),
2) Weigh out 106 g of lanthanum nitrate solution (22% La solution)
3) Mix solution 1 and 2 and dilute with DI water to 1.33 Liters.
4) Water heel of 2 liters and set mixing speed at 400 RPM.
5) Weigh out 191 g of V-250 UOP alumina powder and slowly add into heel. Keep agitation until alumina powder is totally dispersed in the slurry.
6) Weigh out 750 g of soda ash, dissolve in 2.75 liters in DI water. Weigh out the solution before and after precipitation to get the amount of soda ash consumed.
7) Keep the slurry pH=7.0 by adjusting base solution addition rate (room temperature)
8) Precipitation completed by adding copper nitrate and base solution simultaneously in one hour
9) Measure slurry particle size throughout the precipitation
10) Filter the slurry and wash the filter cake
11) Dry the washed filter cake to form powder
12) Calcine the dry powder at 500° C. for 2 hours

Analytical results showed that the resulting catalyst had the following composition: 63.3% CuO-6.3% La₂O₃-29.4% Al₂O₃.

Tablets were prepared from the dry powder. Each tablet had a size of 3 mm×3 mm and were made by adding 2-3% graphite into the dry powder calcined at 500° C. The tablets were finally calcined at 750° C. for 4 h. Each tablet had the following properties: side crush strength (lbs): 27, BET surface area (m²/g): 48 and average bed density (g/mL): 1.45.

Pore size and their distribution and surface area and their distribution for Examples 6-9 were analyzed by nitrogen. These two sets of samples were calcined at different temperatures, 500° C. and 750° C. Higher temperature calcination resulted in lower surface area and larger pore size, and pore area also shifted to larger pore sizes. Pore volume decreased from 0.36 ml/g to 0.30 ml/g when calcination temperature increased from 500° C. to 750° C. This resulted in larger average pore size, from 126 Å to 155 to 160 Å.

Example 7—Chromium-Free Copper Catalyst Composition—Cu—La—Al₂O₃ (Mid La, 750° C. Calcined 4 hr)

A chromium-free copper catalyst composition (Cu—La—Al₂O₃, mid La) was prepared using the same precipitation and drying methods set forth in Example 6. The dry powder from Example 6 was calcined at different temperatures: 600° C., 700° C., 750° C. and 800° C. for 4hours. The process resulted in the following catalysts:
Example 7-1, 600° C.
Example 7-2, 700° C.
Example 7-3, 750° C.
Example 7-4, 800° C.

These catalysts were tested for catalytic performance and analyzed by XRD.

### Example 8—Chromium-Free Copper Catalyst Composition—Cu—La—Al₂O₃ (High La)

The following laboratory procedure was used to prepare a Cu—La—Al₂O₃ (High La) catalyst composition according to embodiments described herein:
1) Weigh out 1400 g of Cu(NO₃)₂ Soln. (16.16% Cu, from plant),
2) Weigh out 212 g of lanthanum nitrate solution (22% La solution)
3) Mix solution 1 and 2 and dilute with DI water to 1.33 Liters.
4) Water heel of 2 liters and set mixing speed at 400 RPM.
5) Weigh out 191 g of V-250 UOP alumina powder and slowly add into heel. Keep agitation until alumina powder is totally dispersed in the slurry.
6) Weigh out 750 g of soda ash, dissolve in 2.75 liters of DI water. Weigh the solution before and after precipitation to get the amount of soda ash consumed.
7) Keep the slurry pH=7.0 by adjusting base solution addition rate (room temperature)
8) Precipitation completed by adding copper nitrate and base solution simultaneously in one hour
9) Measure slurry particle size throughout the precipitation
10) Filter the slurry and wash the filter cake
11) Dry the washed filter cake to form powder
12) Calcine the dry powder at 500° C. for 2 hours

Analytical results show that this catalyst has the following composition: 58.7% CuO, 11.9% La₂O₃ and 28.4% Al₂O₃.

### Example 9—Chromium-Free Copper Catalyst Composition—Cu—La—Al₂O₃ (High La)

A chromium-free copper catalyst composition was prepared using the method set forth in Example 8. The resulting dry powder was calcined at 750° C. for 2 hours. Analytical results show this catalyst has the same compositions as Example 8:58.7% CuO, 11.9% La₂O₃ and 28.4% Al₂O₃.

Pore size and their distribution and surface area and their distribution for Examples 6-9 were analyzed by nitrogen. These two sets of samples were calcined at different temperatures, 500° C. and 750° C. The pore area of samples calcined at 750° C. shifted from about 20 Å to 100 Å to about 100-150 Å. The data are summarized in Table 1.

TABLE 1
Properties of the Chromium-Free Catalysts of Examples 6-9

| | | Example | Example | Example | Example |
|---|---|---|---|---|---|
| | | 6 | 7 | 8 | 9 |
| Surface Area | | | | | |
| Single point surface area | m²/g | 115.1 | 72.9 | 108.2 | 73.3 |
| at P/Po = 0.200963093: | | | | | |
| BET Surface Area: | m²/g | 118.5 | 74.8 | 111.5 | 75.3 |
| Pore Volume | | | | | |
| Single point adsorption | | | | | |
| total pore volume of pores | | | | | |
| less than 2,530.533 Å diameter | cm³/g | 0.4 | 0.3 | 0.4 | 0.3 |
| at P/Po = 0.992311252: | | | | | |
| t-Plot micropore volume: | cm³/g | 0.006 | 0.004 | 0.005 | 0.004 |
| BJH Desorption cumulative | | | | | |
| volume of pores | | | | | |
| between 20.000 Å and | cm³/g | 0.4 | 0.3 | 0.3 | 0.3 |
| 600.000 Å diameter: | | | | | |
| Pore Size | | | | | |
| Adsorption average | Å | 126.5 | 159.9 | 126.8 | 155.9 |
| pore diameter | | | | | |
| (4V/A by BET): | | | | | |

### Incremental and Cumulative Pore Volume vs. Pore Diameter for Selected Catalysts from Examples 6, 7, 8 and 9

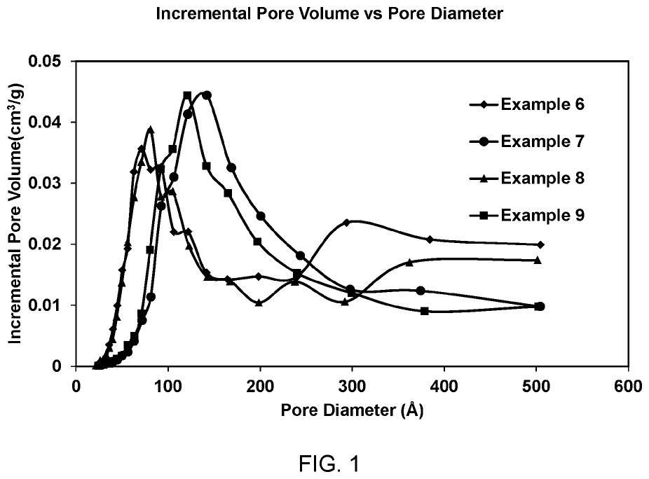

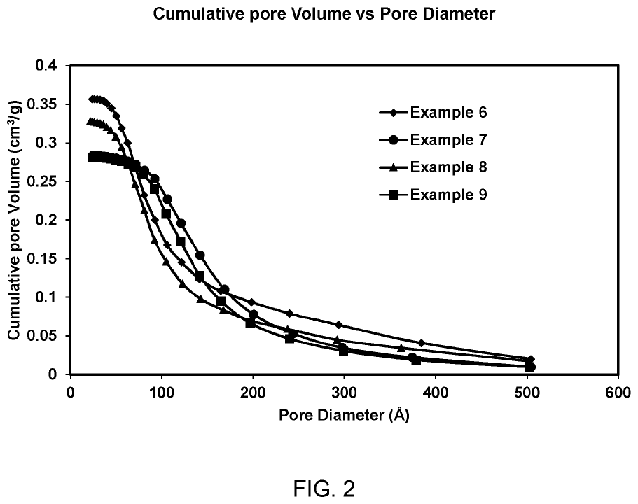

FIG. 1 is a chart showing the incremental pore volume as a function of pore diameter for exemplary catalysts calcined at 500° C. (Examples 6 and 8) and 750° C. (Examples 7-3 and 8), respectively, in accordance with embodiments herein prepared in Examples 6-9. FIG. 2 is a chart showing the cumulative pore area as a function of pore diameter for exemplary catalysts samples calcined at 500° C. (Examples 6 and 8) and 750° C. (Examples 7-3 and 8), respectively, in accordance with embodiments herein prepared in Examples 6-9.

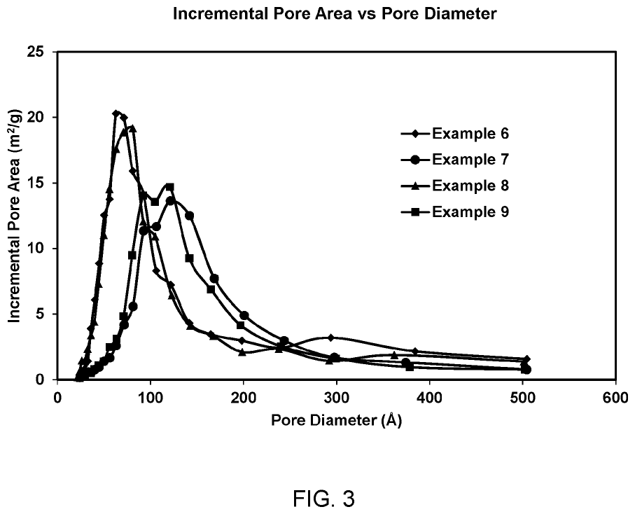

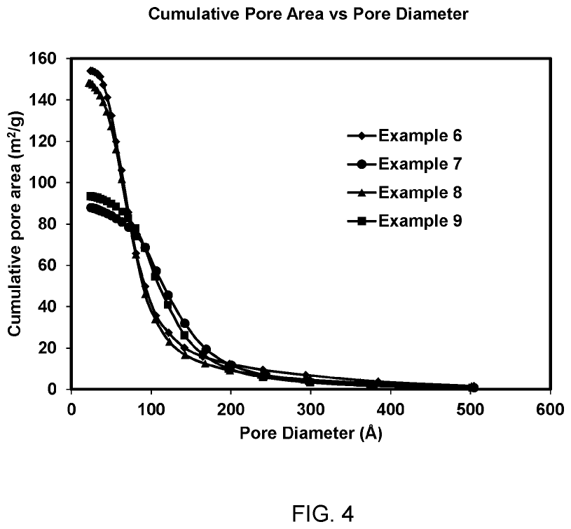

FIG. 3 is a chart showing the incremental pore volume as a function of pore diameter for exemplary catalysts calcined at 500° C. (Examples 6 and 8) and 750° C. (Examples 7-3 and 8), respectively, in accordance with embodiments herein prepared in Examples 6-9. FIG. 4 is a chart showing the cumulative pore area as a function of pore diameter for exemplary catalysts calcined at 500° C. (Examples 6 and 8) and 750° C. (Examples 7-3 and 8), respectively, in accordance with embodiments herein prepared in Examples 6-9. Adding more La, the total pore volume does not change, though a higher calcination temperature decreases the pore volume with relatively larger pore diameter.

### XRD Analysis of Selected Catalysts from Examples 6, 7, 8 and 9

XRD analysis of current inventive catalysts (calcined from 500° C. to 800° C.) were performed to identify the crystallite phases and crystallite sizes.

XRD analysis were performed according to the procedure described here. An Empyrean diffraction system with a copper anode tube was operated with generator settings at 45 kV and 40 mA to produce Cu Kal radiation of wavelength 1.54060 Å used to generate XRD analytical data. The optical path consisted of a 0.04 rad primary soller slit, 15 mm beam mask, 1° divergence slit, 2° anti-scatter slit, the sample, a monochromator, a secondary 0.02 rad soller slit and an X'Celerator position sensitive detector.

The sample was ground to a fine powder using mortar and pestle and then backpacked into a round mount sample holder. The sample holder is loaded onto a sample spinner during data acquisition to improve particle counting statistics. The data collection from the round mount covered a range from 15° to 90° 2θ using a continuous scan with a step size of 0.017° 2θ and a time per step of 400s. A graphite monochromator was used to strip unwanted radiation, including Cu K_{β} radiation.

Panalytical HighScore version 4.5 software and ICDD PDF 4+2020 version powder diffraction file database was used for phase identification analysis. Highscore was also used for profile fitting to determine d-spacing, FWHM and peak positions used to calculate crystallite size estimates using the Scherrer equation.

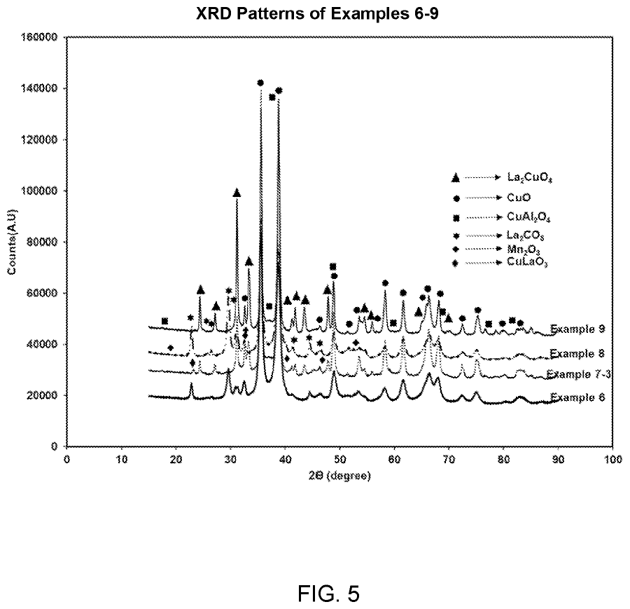

The details of XRD patterns and crystallite size of sample from each example are shown in FIG. 5 and summarized below:

Example 6—Possible phase candidates include monoclinic copper (II) oxide (CuO) and monoclinic lanthanum oxide carbonate (La₂CO₅).

Example 7—Possible phase candidates include monoclinic copper (II) oxide (CuO), tetragonal copper lanthanum oxide (CuLaO₃) and orthorhombic lanthanum copper oxide (La₂CuO₄). and cubic copper aluminum oxide (CuAl₂O₄) may also be present.

Example 8—Possible phase candidates include monoclinic copper (II) oxide (CuO), monoclinic lanthanum oxide carbonate (La₂CO₅) and possibly cubic spinel manganese oxide (Mn₂O₃).

Example 9—Possible phase candidates include monoclinic copper (II) oxide (CuO), orthorhombic lanthanum copper oxide (La₂CuO₄) and possibly cubic copper aluminum oxide (CuAl₂O₄) forming.

FIG. 5 is a chart showing the x-ray diffraction (XRD) patterns for catalysts calcined at 500° C. (Examples 6 and 8) and 750° C. (Examples 7-3 and 8), respectively, in accordance with embodiments herein prepared in Examples 6-9.

As calcination temperature increases, La₂CO₅ transforms to tetragonal copper lanthanum oxide (CuLaO₃) or orthorhombic lanthanum copper oxide (La₂CuO₄; and part of CuO transforms to tetragonal copper lanthanum oxide (CuLaO₃) and orthorhombic lanthanum copper oxide (La₂CuO₄) and possibly cubic copper aluminum oxide (CuAl₂O₄).

The Crystallite phase identification of each of these samples was compared to the reference XRD index card (Tables 2-6). Tables 2-6 provide the 2θ, d-spacing, I (relative intensity), hkl (crystal faces). By comparing the XRD diffraction patterns with the 2θ and relative intensity, the crystallite phase characteristics for each chromium-free copper catalyst sample was identified.

TABLE 2
Crystallite phase characteristics for CuO 00-048-1548
d-spacings (55) - Cu O - 00-048-1548 (Stick, Fixed
Slit Intensity) - X-ray (Cu Kα1 1.54056 Å)

| 2θ (°) | d (Å) | I | h | k | l | * |
|---|---|---|---|---|---|---|
| 32.508 | 2.752010 | 13 | 1 | 1 | 0 | |
| 35.417 | 2.532360 | 37 | 0 | 0 | 2 | |
| 35.543 | 2.523670 | 100 | 1 | 1 | −1 | |
| 38.708 | 2.324290 | 99 | 1 | 1 | 1 | |
| 38.902 | 2.313150 | 21 | 2 | 0 | 0 | |
| 46.259 | 1.960950 | 3 | 1 | 1 | −2 | |
| 48.716 | 1.867640 | 30 | 2 | 0 | −2 | |
| 51.343 | 1.778080 | 1 | 1 | 1 | 2 | |
| 53.485 | 1.711790 | 7 | 0 | 2 | 0 | |
| 56.741 | 1.621050 | <1 | 0 | 2 | 1 | |
| 58.264 | 1.582270 | 10 | 2 | 0 | 2 | |
| 61.524 | 1.506000 | 20 | 1 | 1 | −3 | |
| 65.811 | 1.417890 | 10 | 0 | 2 | 2 | |
| 66.220 | 1.410130 | 15 | 3 | 1 | −1 | |
| 66.447 | 1.405860 | <1 | 3 | 1 | 0 | |
| 67.903 | 1.379220 | 6 | 1 | 1 | 3 | |
| 68.123 | 1.375300 | 14 | 2 | 2 | 0 | |
| 68.905 | 1.361580 | <1 | 2 | 2 | −1 | |
| 71.681 | 1.315520 | <1 | 3 | 1 | −2 | |
| 72.371 | 1.304670 | 5 | 3 | 1 | 1 | |
| 72.942 | 1.295860 | <1 | 2 | 2 | 1 | |
| 74.976 | 1.265670 | 6 | 0 | 0 | 4 | |
| 75.243 | 1.261840 | 5 | 2 | 2 | −2 | |
| 79.731 | 1.201710 | <1 | 0 | 2 | 3 | |
| 80.155 | 1.196420 | 2 | 2 | 0 | −4 | |
| 80.239 | 1.195380 | <1 | 1 | 1 | −4 | |
| 82.359 | 1.169890 | 4 | 3 | 1 | −3 | |
| 83.063 | 1.161760 | 2 | 2 | 2 | 2 | |
| 83.566 | 1.156040 | 2 m | 3 | 1 | 2 | |
| 83.566 | 1.156040 | 2 m | 4 | 0 | 0 | |
| 86.530 | 1.123880 | 1 | 4 | 0 | −2 | |
| 86.772 | 1.121370 | <1 | 2 | 2 | −3 | |
| 87.965 | 1.109210 | <1 | 1 | 1 | 4 | |
| 88.051 | 1.108350 | <1 | 1 | 3 | 0 | |
| 89.787 | 1.091370 | 3 | 1 | 3 | −1 | |
| 91.726 | 1.073300 | 1 | 1 | 3 | 1 | |
| 95.562 | 1.040100 | <1 | 2 | 0 | 4 | |
| 98.388 | 1.017640 | 1 m | 0 | 2 | 4 | |
| 98.388 | 1.017640 | 1 m | 2 | 2 | 3 | |
| 99.681 | 1.007890 | 2 | 3 | 1 | 3 | |
| 101.932 | 0.991645 | <1 | 4 | 0 | 2 | |
| 103.353 | 0.981843 | 2 | 1 | 1 | −5 | |
| 103.561 | 0.980438 | 2 | 2 | 2 | −4 | |
| 107.045 | 0.957951 | 2 | 4 | 2 | 0 | |
| 109.515 | 0.943142 | <1 | 1 | 3 | −3 | |

| 2θ (°) | d (Å) | I | h | k | l |
|---|---|---|---|---|---|
| 110.166 | 0.939387 | 3 | 4 | 2 | −2 |
| 111.187 | 0.933618 | <1 | 4 | 0 | −4 |
| 113.485 | 0.921153 | 2 | 1 | 1 | 5 |
| 114.056 | 0.918164 | <1 m | 3 | 3 | −1 |
| 114.056 | 0.918164 | <1 m | 4 | 2 | 1 |
| 115.739 | 0.909592 | 1 | 1 | 3 | 3 |
| 116.833 | 0.904212 | <1 | 5 | 1 | −1 |
| 120.216 | 0.888477 | 1 | 2 | 2 | 4 |
| 120.504 | 0.887198 | 3 m | 3 | 1 | −5 |
| 120.504 | 0.887198 | 3 m | 3 | 3 | 1 |

TABLE 3
Crystallite phase characteristics for CuAl₂O₄ 04-002-5874
d-spacings (31) - Cu Al2 O4 - 04-002-5874 (Stick, Fixed
Slit Intensity) - X-ray (Cu Kα1 1.54056 Å)

| 2θ (°) | d (Å) | I | h | k | l | * |
|---|---|---|---|---|---|---|
| 18.961 | 4.676540 | 37 | 1 | 1 | 1 | |
| 31.206 | 2.863780 | 471 | 2 | 2 | 0 | |
| 36.770 | 2.442240 | 1000 | 3 | 1 | 1 | |
| 38.468 | 2.338270 | 3 | 2 | 2 | 2 | |
| 44.715 | 2.025000 | 187 | 4 | 0 | 0 | |
| 48.978 | 1.858270 | 10 | 3 | 3 | 1 | |
| 55.534 | 1.653410 | 118 | 4 | 2 | 2 | |
| 59.225 | 1.558850 | 327 | 5 | 1 | 1 | |
| 65.088 | 1.431890 | 400 | 4 | 4 | 0 | |
| 68.471 | 1.369150 | 2 | 5 | 3 | 1 | |
| 69.581 | 1.350000 | 1 | 4 | 4 | 2 | |
| 73.947 | 1.280720 | 33 | 6 | 2 | 0 | |
| 77.157 | 1.235240 | 67 | 5 | 3 | 3 | |
| 78.218 | 1.221120 | 10 | 6 | 2 | 2 | |
| 82.424 | 1.169130 | 16 | 4 | 4 | 4 | |
| 85.550 | 1.134230 | 5 | 7 | 1 | 1 | |
| 90.736 | 1.082410 | 39 | 6 | 4 | 2 | |
| 93.848 | 1.054530 | 83 | 7 | 3 | 1 | |
| 99.064 | 1.012500 | 36 | 8 | 0 | 0 | |
| 102.228 | 0.989573 | 2 | 7 | 3 | 3 | |
| 103.291 | 0.982269 | 1 | 6 | 4 | 4 | |
| 107.592 | 0.954594 | 18 | 8 | 2 | 2 | |
| 110.885 | 0.935308 | 50 | 7 | 5 | 1 | |
| 111.998 | 0.929134 | 5 | 6 | 6 | 2 | |
| 116.547 | 0.905608 | 15 | 8 | 4 | 0 | |
| 120.079 | 0.889091 | 2 | 9 | 1 | 1 | |
| 121.284 | 0.883783 | 1 | 8 | 4 | 2 | |
| 126.272 | 0.863463 | 5 | 6 | 6 | 4 | |
| 130.232 | 0.849111 | 42 | 9 | 3 | 1 | |
| 137.418 | 0.826703 | 73 | 8 | 4 | 4 | |
| 142.239 | 0.814081 | 1 | 7 | 7 | 1 | |

TABLE 4
Crystallite phase characteristics for La₂CO₅ 00-023-0322
d-spacings (15) - La2 C O5 - 00-023-0322 (Stick, Fixed
Slit Intensity) - X-ray (Cu Kα1 1.54056 Å)

| 2θ (°) | d (Å) | I | h | k | l | * |
|---|---|---|---|---|---|---|
| 13.105 | 6.750 | 25 | 0 | 0 | 2 | |
| 22.842 | 3.890 | 45 | 0 | 1 | 1 | |
| 25.502 | 3.490 | 4 | 0 | 1 | 2 | |
| 26.426 | 3.370 | 6 | 0 | 0 | 4 | |
| 29.554 | 3.020 | 100 | 0 | 1 | 3 | |
| 30.829 | 2.898 | 25 | 1 | 1 | 0 | |
| 31.327 | 2.853 | 25 | −1 | 1 | 1 | |
| 32.436 | 2.758 | 2 | | | | |
| 33.652 | 2.661 | 2 | −1 | 1 | 2 | |
| 34.088 | 2.628 | 2 | 1 | 1 | 2 | |

| 2θ (°) | d (Å) | I | h | k | l |
|---|---|---|---|---|---|
| 37.344 | 2.406 | 2 | 1 | 1 | 3 |
| 40.096 | 2.247 | 14 | 0 | 0 | 6 |
| 41.166 | 2.191 | 25 | −1 | 1 | 4 |
| 44.507 | 2.034 | 20 | 0 | 2 | 0 |
| 46.559 | 1.949 | 6 | 0 | 2 | 2 |

TABLE 5
Crystallite phase characteristics for CuLaO₃, 04-022-5441
d-spacings (52) - Cu La O3 - 04-022-5441 (Stick, Fixed
Slit Intensity) - X-ray (Cu Kα1 1.54056 Å)

| 2θ (°) | d (Å) | I | h | k | l | * |
|---|---|---|---|---|---|---|
| 22.350 | 3.972700 | 57 | 0 | 0 | 1 | |
| 23.274 | 3.818750 | 102 | 1 | 0 | 0 | |
| 32.495 | 2.753080 | 1000 | 1 | 0 | 1 | |
| 33.149 | 2.700260 | 478 | 1 | 1 | 0 | |
| 40.354 | 2.233230 | 222 | 1 | 1 | 1 | |
| 45.634 | 1.986350 | 156 | 0 | 0 | 2 | |
| 47.584 | 1.909370 | 269 | 2 | 0 | 0 | |
| 51.839 | 1.762210 | 25 | 1 | 0 | 2 | |
| 53.179 | 1.720930 | 23 | 2 | 0 | 1 | |
| 53.620 | 1.707800 | 23 | 2 | 1 | 0 | |
| 57.555 | 1.600060 | 153 | 1 | 1 | 2 | |
| 58.806 | 1.568970 | 284 | 2 | 1 | 1 | |
| 68.053 | 1.376540 | 132 | 2 | 0 | 2 | |
| 69.573 | 1.350130 | 61 | 2 | 2 | 0 | |
| 71.138 | 1.324230 | 2 | 0 | 0 | 3 | |
| 73.000 | 1.294980 | 18 | 2 | 1 | 2 | |
| 74.108 | 1.278330 | 9 | 2 | 2 | 1 | |
| 74.476 | 1.272920 | 4 | 3 | 0 | 0 | |
| 76.000 | 1.251140 | 54 | 1 | 0 | 3 | |
| 78.904 | 1.212210 | 47 | 3 | 0 | 1 | |
| 79.266 | 1.207590 | 47 | 3 | 1 | 0 | |
| 80.761 | 1.188960 | 18 | 1 | 1 | 3 | |
| 83.623 | 1.155390 | 31 | 3 | 1 | 1 | |
| 87.235 | 1.116610 | 51 | 2 | 2 | 2 | |
| 90.125 | 1.088150 | 5 | 2 | 0 | 3 | |
| 91.898 | 1.071740 | 5 | 3 | 0 | 2 | |
| 93.317 | 1.059130 | 5 | 3 | 2 | 0 | |
| 94.794 | 1.046490 | 50 | 2 | 1 | 3 | |
| 96.574 | 1.031870 | 48 | 3 | 1 | 2 | |
| 97.645 | 1.023390 | 47 | 3 | 2 | 1 | |
| 101.714 | 0.993175 | 8 | 0 | 0 | 4 | |
| 106.523 | 0.961199 | 4 | 1 | 0 | 4 | |
| 107.577 | 0.954687 | 14 | 4 | 0 | 0 | |
| 109.129 | 0.945398 | 4 | 2 | 2 | 3 | |
| 111.015 | 0.934577 | 7 | 3 | 2 | 2 | |
| 111.455 | 0.932125 | 17 | 1 | 1 | 4 | |
| 112.159 | 0.928260 | 4 | 4 | 0 | 1 | |
| 112.542 | 0.926183 | 4 | 4 | 1 | 0 | |
| 114.146 | 0.917694 | 17 | 3 | 0 | 3 | |
| 117.293 | 0.901994 | 32 | 4 | 1 | 1 | |
| 117.693 | 0.900088 | 9 | 3 | 3 | 0 | |
| 119.370 | 0.892290 | 14 | 3 | 1 | 3 | |
| 121.906 | 0.881105 | 23 | 2 | 0 | 4 | |
| 122.678 | 0.877839 | 7 | 3 | 3 | 1 | |
| 127.066 | 0.860463 | 22 | 4 | 0 | 2 | |

| 2θ (°) | d (Å) | I | h | k | l |
|---|---|---|---|---|---|
| 127.582 | 0.858548 | 7 | 2 | 1 | 4 |
| 128.864 | 0.853898 | 22 | 4 | 2 | 0 |
| 133.166 | 0.839418 | 6 | 4 | 1 | 2 |
| 134.641 | 0.834832 | 6 | 4 | 2 | 1 |
| 137.270 | 0.827120 | 27 | 3 | 2 | 3 |
| 139.950 | 0.819844 | 13 | 3 | 3 | 2 |
| 148.651 | 0.800031 | 19 | 2 | 2 | 4 |

TABLE 6
Crystallite phase characteristics for La₂CuO₄ 00-038-0709
d-spacings (26) - La2 Cu O4 - 00-038-0709 (Stick, Fixed
Slit Intensity) - X-ray (Cu Kα1 1.54056 Å)

| 2θ (°) | d (Å) | I | h | k | l | * |
|---|---|---|---|---|---|---|
| 13.404 | 6.6000 | 4 | 0 | 0 | 2 | |
| 24.332 | 3.6550 | 24 | 1 | 1 | 1 | |
| 27.097 | 3.2880 | 13 | 0 | 0 | 4 | |
| 31.115 | 2.8720 | 100 | 1 | 1 | 3 | |
| 33.164 | 2.6991 | 29 | 0 | 2 | 0 | |
| 33.443 | 2.6772 | 28 | 2 | 0 | 0 | |
| 41.166 | 2.1910 | 10 | 0 | 0 | 6 | |
| 41.716 | 2.1634 | 20 | 1 | 1 | 5 | |
| 43.334 | 2.0863 | 15 | 0 | 2 | 4 | |
| 43.544 | 2.0767 | 15 | 2 | 0 | 4 | |
| 47.799 | 1.9013 | 27 | 2 | 2 | 0 | |
| 53.825 | 1.7018 | 6 | 0 | 2 | 6 | |
| 54.003 | 1.6966 | 7 | 2 | 0 | 6 | |
| 54.145 | 1.6925 | 6 | 1 | 3 | 1 | |
| 54.433 | 1.6842 | 11 | 1 | 1 | 7 | |
| 54.542 | 1.6811 | 8 | 3 | 1 | 1 | |
| 55.882 | 1.6461 | 8 | 2 | 2 | 4 | |
| 55.890 | 1.6437 | 9 | 0 | 0 | 8 | |

| 2θ (°) | d (Å) | I | h | k | l |
|---|---|---|---|---|---|
| 57.957 | 1.5899 | 12 | 1 | 3 | 3 |
| 58.319 | 1.5809 | 12 | 3 | 1 | 3 |
| 64.899 | 1.4356 | 7 | 2 | 2 | 6 |
| 65.077 | 1.4321 | 7 | 1 | 3 | 5 |
| 65.457 | 1.4247 | 4 | 3 | 1 | 5 |
| 66.568 | 1.4036 | 4 | 0 | 2 | 8 |
| 66.729 | 1.4006 | 4 | 2 | 0 | 8 |
| 69.557 | 1.3504 | 2 | 0 | 4 | 0 |

### Example 10—Chromium-free Copper Catalyst Composition—Cu—Mn—La—Al₂O₃ (500° C.) Targeted 58% CuO-3% Mn₂O₃-9% La₂O₃-30% Al₂O₃

The following laboratory procedure was used to prepare a Cu—Mn—La—Al₂O₃ (36084-4A (500° C.) (target 58% CuO-3% Mn₂O₃-9% La₂O₃-30% Al₂O₃) catalyst composition according to embodiments described herein:
1) Weigh out 930 g of Cu(NO₃)₂ soln. (16.16% Cu, from plant),
2) Weigh out 53 g of manganese nitrate solution
3) Weigh out 115.5 g of lanthanum nitrate solution (22% La solution)
4) Mix solution 1, 2 and 3 and dilute with DI water to 1.0 Liter.
5) Water heel of 1.33 liters and set mixing speed at 400 RPM.
6) Weigh out 142 g of V-250 UOP alumina powder and slowly add into heel. Keep agitation until alumina powder is totally dispersed in the slurry.
7) Weigh 500 g of soda ash, dissolve in 1.33 liters of DI water. Weigh the solution before and after precipitation to get the amount of soda ash consumed.
8) Keep the slurry pH=7.0 by adjusting base solution addition rate (room temperature)
9) Precipitation completed by adding copper nitrate and base solution simultaneously in one hour
10) Measure slurry particle size throughout the precipitation
11) Filter the slurry and wash the filter cake
12) Dry the washed filter cake to form powder
13) Calcine the dry powder at 500° C. for 2 hours sample

### Example 11—Chromium-free Copper Catalyst with Targeted Composition (750° C.) 58% CuO-3% Mn₂O₃-9% La₂O₃-30% Al₂O₃

A chromium-free copper catalyst composition (Cu—Mn—La—Al₂O₃, 750° C. targeted 58% CuO-3% MnO-9% La₂O₃-30% Al₂O₃) was prepared using the method set forth in Example 10. The resulting dry powder was calcined at 750° C. for 2 hours sample (750° C.). The catalyst composition had a particle size distribution (microns): D₁₀ 5.76 μm, D₅₀ 18.7 μm and D₉₀ 38.3 μm.

### Example 12—Chromium-free Copper Catalyst with Targeted Composition—Cu—Mn—La—Al₂O₃ (500° C.) 58% CuO-6% Mn₂O₃-6% La₂O₃-30% Al₂O₃

The following laboratory procedure was used to prepare a Cu—Mn—La—Al₂O₃ (500° C.) targeted 58% CuO-6% Mn₂O₃-6% La₂O₃-30% Al₂O₃ catalyst composition according to embodiments described herein:
1) Weigh out 930 g of Cu(NO₃)₂ soln. (16.16% Cu, from plant),
2) Weigh out 105 g of manganese nitrate solution
3) Weigh out 77 g of lanthanum nitrate solution (22% La solution)
4) Mix solution 1 and 2 and dilute with DI water to 1.0 Liters.
5) Water heel of 1.33 liters and set mixing speed at 400 RPM.
6) Weigh out 142 g of V-250 UOP alumina powder and slowly add into heel. Keep agitation until alumina powder is totally dispersed in the slurry.
7) Weigh out 500 g of soda ash, dissolve in D.I. dilute to 1.33 liters. Weigh the solution before and after precipitation to get the amount of soda ash consumed.
8) Keep the slurry pH=7.0˜7.5 by adjusting base solution addition rate (room temperature)
9) Precipitation completed by adding copper nitrate and base solution simultaneously in one hour
10) Measure slurry particle size throughout the precipitation
11) Filter the slurry and wash the filter cake
12) Dry the washed filter cake to form powder
13) Calcine the dry powder at 500° C. for 2 hours

### Example 13—Chromium-Free Copper Catalyst with Targeted Composition (750° C.) 58% CuO-6% Mn₂O₃-6% La₂O₃-30% Al₂O₃

A chromium-free copper catalyst composition (Cu—Mn—La—Al₂O₃ (750° C.) targeted 58% CuO-6% Mn₂O₃-6% La₂O₃-30% Al₂O₃) was prepared using the method set forth in Example 12. The resulting dry powder was calcined at 750° C. for 2 hours sample. The catalyst composition had a particle size distribution (microns): D₁₀ 6.4 μm, D₅₀ 21 μm and D₉₀ 41.5 μm.

### Example 14—Chromium-Free Copper Catalyst with Targeted Composition—(500° C.) 58% CuO-9% Mn₂O₃-3% La₂O₃-30% Al₂O₃

The following laboratory procedure was used to prepare a Cu—Mn—La—Al₂O₃ 36084-12A (500° C.) targeted for 58% CuO-9% Mn₂O₃-3% La₂O₃-30% Al₂O₃ catalyst composition according to embodiments described herein:
1) Weigh out 930 g of Cu(NO₃)₂ soln. (16.16% Cu, from plant),
2) Weigh out 157 g of manganese nitrate solution
3) Weigh out 39 g of lanthanum nitrate solution (22% La solution)
4) Mix solution 1 and 2 and dilute with DI water to 1.0 Liters.
5) Water heel of 1.33 liters and set mixing speed at 400 RPM.
6) Weigh out 142 g of V-250 UOP alumina powder and slowly add into heel. Keep agitation until alumina powder is totally dispersed in the slurry.
7) Weigh out 500 g of soda ash, dissolve in D.I. dilute to 1.33 liters. Weigh the solution before and after precipitation to get the amount of soda ash consumed.
8) Keep the slurry pH=7.0 by adjusting base solution addition rate (room temperature)
9) Precipitation completed by adding copper nitrate and base solution simultaneously in one hour
10) Measure slurry particle size throughout the precipitation
11) Filter the slurry and wash the filter cake
12) Dry the washed filter cake to form powder
13) Calcine the dry powder at 500° C. for 2 hours sample 36084-12A (500° C.)

### Example 15—Chromium-Free Copper Catalyst Targeted Composition (750° C.) 58% CuO-9% MnO-3% La₂O₃-30% Al₂O₃

A chromium-free copper catalyst composition (Cu—Mn—La—Al₂O₃, 750° C., targeted for 58% CuO-9% Mn₂O₃-3% La₂O₃-30% Al₂O₃) was prepared using the method set forth in Example 14. The resulting dry powder was calcined at 750° C. for 2 hours. The catalyst composition had a particle size distribution (microns): D₁₀ 8.54 μm, D₅₀ 19.5 μm and D₉₀ 35.1 μm.

Further analyses of the CuO crystallite size of the catalyst prepared in various examples showed the crystallite size of CuO was from about 90 Å to about 260 Å. The results are summarized in Table 7. Using dry powder from Example 6, the powder was subjected to calcination at different temperatures. X-ray diffraction (XRD) analysis showed that the crystalline phases changed as the temperature increased. The data showed that La, at low temperature, is in the form of lanthanum oxide carbonate, and it transforms to orthorhombic lanthanum copper oxide (La₂CuO₄ at 700° C., then partially to tetragonal copper lanthanum oxide (CuLaO₃) at 750° C., and finally, in addition to the phases at 750° C., a new phase of cubic copper aluminum oxide (CuAl₂O₄) was formed at 800° C.

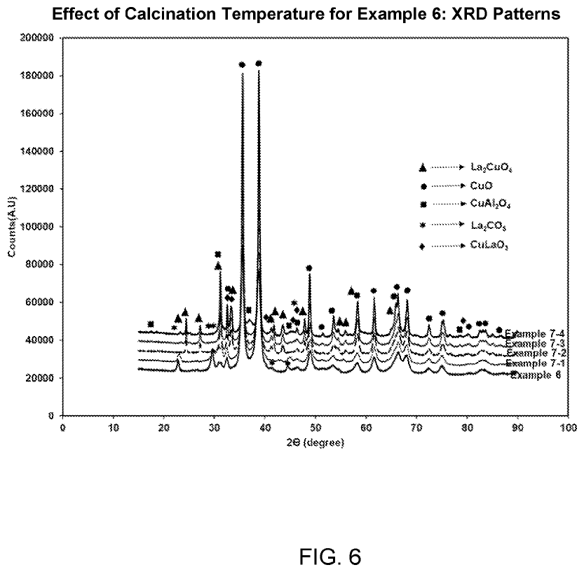

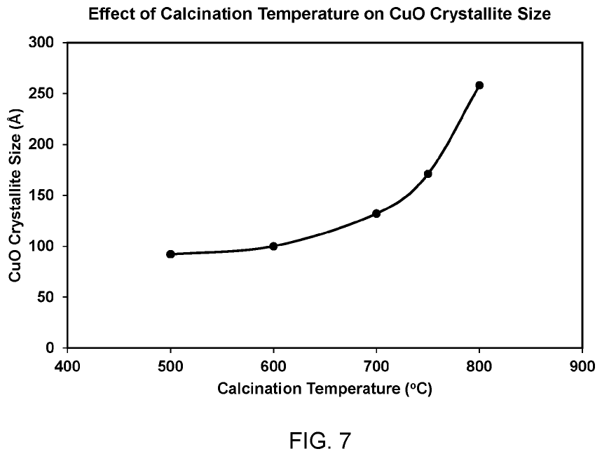

The XRD patterns are shown in FIG. 6. FIG. 6 is a chart showing the XRD patterns for catalysts prepared using dry powder from Example 6, calcined at different temperatures, in accordance with embodiments herein prepared for Examples 6 and 7-1 to 7-4. Analysis of the CuO crystallite size showed CuO size growth with calcination temperatures, changing from 90 Å to 220 Å. The results are summarized in Table 7. FIG. 7 also shows the effect of calcination temperature on CuO crystallite size.

TABLE 7
Analysis of CuO crystallite size as
a function of calcination temperature

| | Calcination | CuO Crystallite |
|---|---|---|
| | Temperature, ° C. | Size, Å |
| Example 6 | 500 | 92 |
| Example 7-1 | 600 | 100 |
| Example 7-2 | 700 | 132 |
| Example 7-3 | 750 | 171 |
| Example 7-4 | 800 | 258 |
| Example 8 | 500 | 102 |
| Example 9 | 750 | 221 |

### Example 16—Catalyst Performance Evaluations

Testing procedure: Catalytic activity and selectivity of catalyst compositions according to embodiments herein and prepared in the above examples, were evaluated by slurry phase hydrogenolysis of methyl ester to fatty alcohol. Catalyst performance evaluations were performed for both methyl ester hydrogenolysis and wax ester hydrogenolysis in one-liter autoclave.

### Procedure of Methyl-Ester Hydrogenolysis Testing (Including Re-Use Test)

Catalyst performance testing was conducted in a 1-liter stainless steel autoclave equipped with baffles and a Disperimax™ impeller. For each experiment, the autoclave was charged with 396 g of fatty acid methyl ester (approx. 75 wt % C₁₂/25% C₁₄-C₁₈) along with catalyst powder at a concentration of 0.8 wt %. Subsequent to 2-3 N₂ pressure purges at 250 psig to remove air followed by 2-3 H₂ pressure purges at 250 psig, the autoclave was heated to 280° C. under 2000 RPM agitation and 150 psig H₂. Once the temperature stabilized at 280° C., agitation was paused and the Hz pressure increased to 2500 psig. The agitation was then restarted at 2000 RPM; this was designated as Time 0. Approximately 5-10 cc of product sample was collected once per hour through 5 hours.

In order to evaluate catalyst deactivation, the experiment can be repeated multiple times using the same catalyst. In this case, after sampling has been completed, the reactor is cooled and depressurized, and the entire liquid contents of the autoclave are drained through a 1.2 μm fritted disk which allows the catalyst to remain in the reactor. Fresh feed is then added to the autoclave and the experiment is repeated in its entirety.

Each sample was analyzed via gas chromatography whereby fatty alcohol, wax ester, and hydrocarbon byproduct were quantified. The gas chromatograph used for the analyses was an Agilent model 7890 equipped with a flame ionization detector (FID) and Quadrex capillary column, 75 μm×320 μm×0.25 μm. Column flow was held constant at 4.7 cc/min at a split ratio of 100:1. Upon sample injection (1 μl), the oven was heated from 100 to 200° C. at 5°/min and held at 200° C. for 55 min.

### Procedure for Wax-Ester Hydrogenolysis

Reaction Conditions: Pressure: 4350 psi; Temperature: 300° C.; Agitation: 1500 rpm Feed: C₁₆-C₁₈ fatty acid (27% C₁₆ acid, 72% C₁₈ acid); Heel: 454 g C₁₂-C₁₄ fatty alcohol; 6 h runs (each hour, injection of 55 g of fatty acid for 0,1,2,3^{rd} h)

Procedure:
1. Load the catalyst (0.75 wt % catalyst loading) through opening the top screw of the reactor head. 454 g of C₁₂-C₁₄ fatty alcohol is loaded through the funnel located on the gas line which is used for pressurization and hydrogen gas feed.
2. Purge the autoclave system with N₂ few times to remove air and then purge with hydrogen few times.
3. Start the agitation to 1500 rpm and start the ramping up the temperature of the furnace, the autoclave is jacketed. Typical ramping rate 3° C./min.
4. When the temperature reaches at 300° C., start pressurization the autoclave with hydrogen to 4350 psi. Inject 55 g of C₁₆-C₁₈ fatty acid through pump. This will start time “0”. 5 ml of liquid sample was collected through the port with the frit at the tip inside the autoclave at 1^{st} hr. Then inject another 55 g of C₁₆-C₁₈ fatty acid. At 2^{nd} and 3^{rd} hour, this sampling and injection of fatty acid will be continued. The experiment was continued to 6 hours without further injection of fatty acid after 3 HOS. Each sample (total 6 samples) was analyzed in GC for the by-products.
5. The SAP value of each sample was calculated by wet titration. The conversion of fatty acid was calculated based on the % SAP value reduction.
6. Conversion, %=(SAP value in resulting feed mixture-SAP value in the product)*100/SAP value in resulting feed mixture

### Comparison of Catalyst Performance of a CuCr Reference Catalyst with CuMnAl₂O₃ and CuLaAl₂O₃ Catalyst Compositions According to Embodiments Herein in Multiple-Cycle Test

### Comparison with a CuCr Reference Catalyst (Example 1)

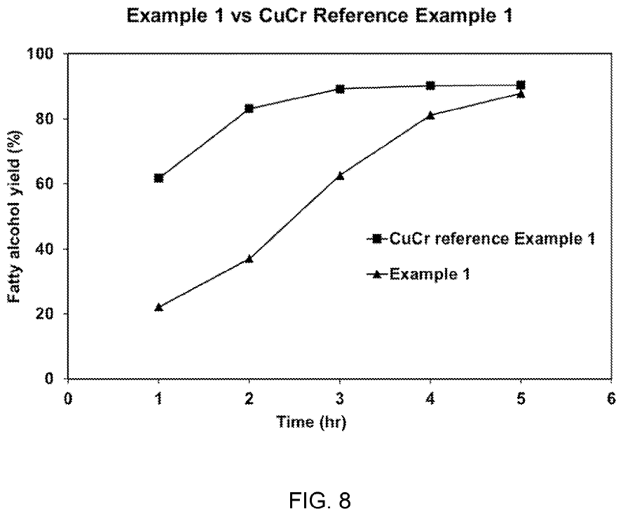

Fatty alcohol yield curves (FIG. 8) showed that when using inventive Cu—Al₂O₃ catalyst compositions prepared by a deposition-precipitation method with V-250 UOP alumina in Example 1 according to embodiments herein, the inventive catalysts had lower alcohol yield than the CuCr reference catalyst throughout six (6) hours of reaction time. This prompted the development of promoted Cu—Al₂O; catalyst by using Mn, La and other metal or metal oxides.

### Comparison of Example 2 Mn promoted CuMn—Al₂O₃ V250 with the CuCr Reference Catalyst (Example 1)

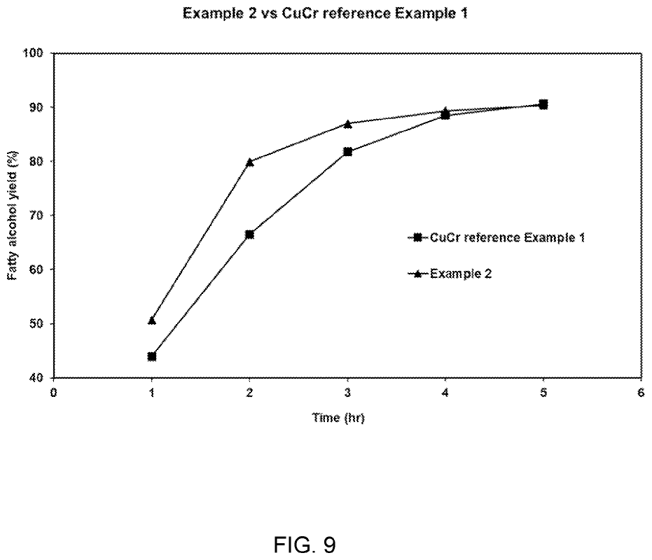

With addition of Mn to Cu—Al₂O₃ using a precipitation-deposition method, the Example 2 catalyst had significantly better alcohol yield than not only the Example 1 catalyst, but also than the CuCr reference catalyst (FIG. 9).

### Example 3 and 4 vs Reference Catalysts

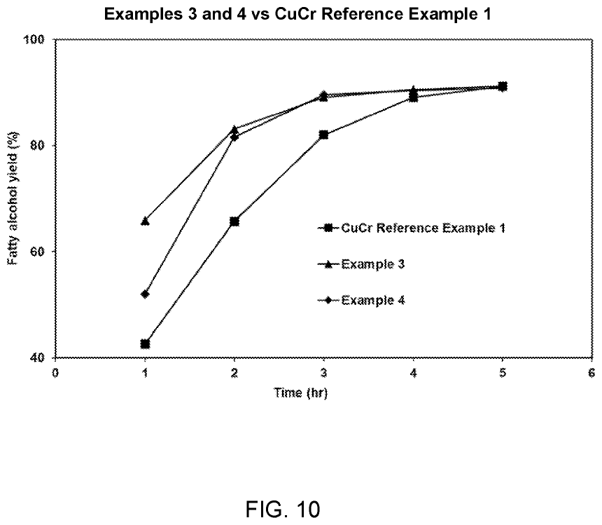

Mn promoted Cu on alumina catalysts from Examples 3 and 4 (calcined at different temperatures) were tested against reference Example 1 (CuCr). The catalytic performance of these two catalysts was better than the commercially available CuCr reference catalyst (FIG. 10).

### Performance of Example 5, 6, and 8 La2O3 Promoted Cu—Al₂O₃ catalysts vs CuCr Reference

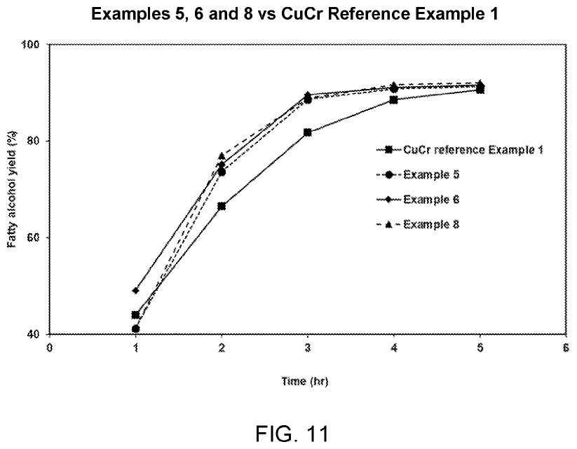

At three different La loadings, all of the catalysts from Examples 5, 6 and 8 all had better alcohol yield than the CuCr reference catalyst throughout the reaction time (FIG. 11). These results demonstrate that catalysts according to embodiments described herein can outperform a commercial CuCr reference catalyst. These examples show that La is a promoter for Cu catalyst on alumina.

### Example 7 vs CuCr Reference

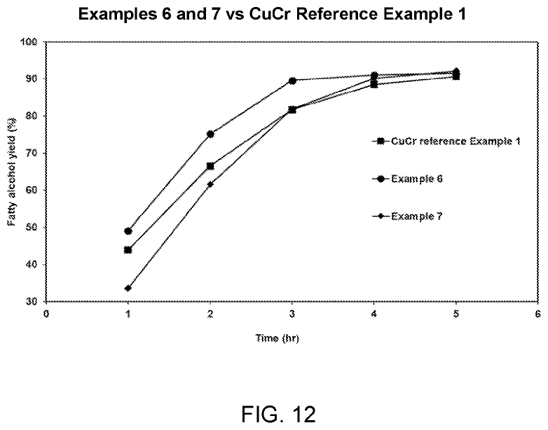

Two new Cr-free catalyst candidates were prepared and tested, Example 7 Cu/La/Al₂O₃, which is similar to Example 6, but calcined at a higher temperature, 750°, performed comparably to the reference CuCr catalyst after 3 hours of reaction time. Fatty alcohol yield curves (FIG. 12) showed that the catalyst composition from Example 7 provided higher alcohol yield than the CuCr reference catalyst when approaching equilibrium at about 5 hours reaction time.

### Example 17—Catalyst Stability Test by Multi-Cycle Performance Test

The catalyst stability as measured by multi-cycle performance test was evaluated. To simulate longevity or useful life of catalysts according to embodiments here, a multiple-cycle performance test provided useful results. Multiple-reuse tests were conducted on two samples (Example 6 and Example 3) with the reference catalyst (Example 1) under standard lab testing conditions. The only difference is that catalyst after each activity test is kept in the reactor for further testing with fresh feed.

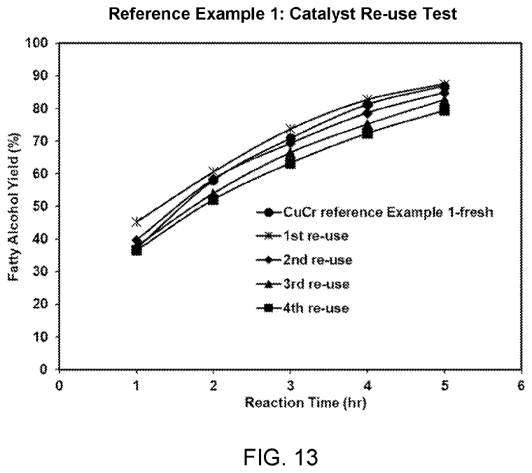

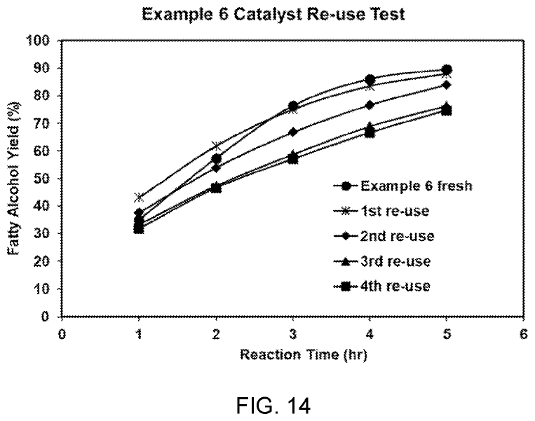

The results are shown in FIGS. 13 and 14. FIG. 13 is a chart showing a multi-cycle performance test for a catalyst composition according to embodiments herein. FIG. 14 is a chart showing a multi-cycle performance test for a catalyst composition according to embodiments herein.

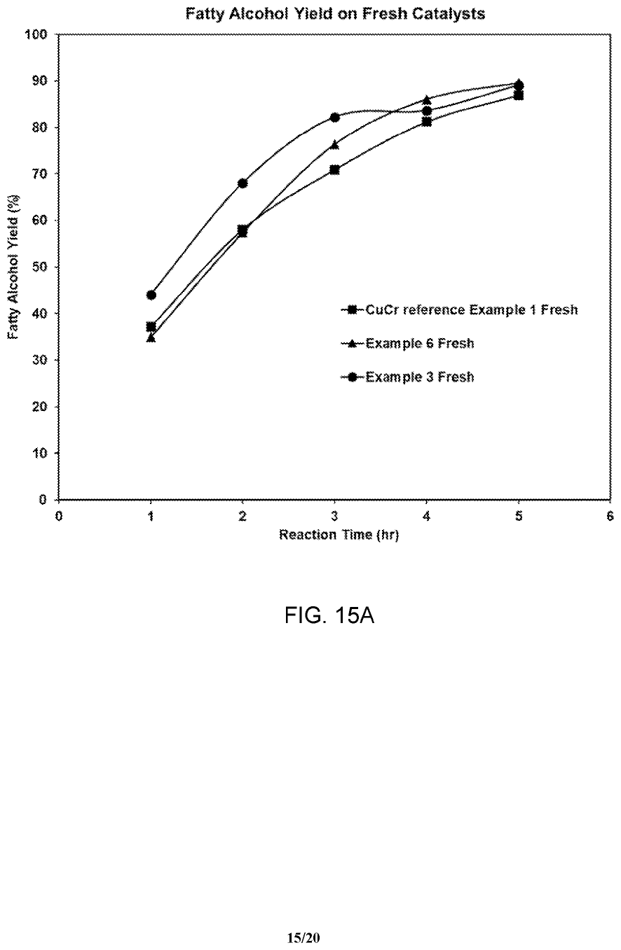

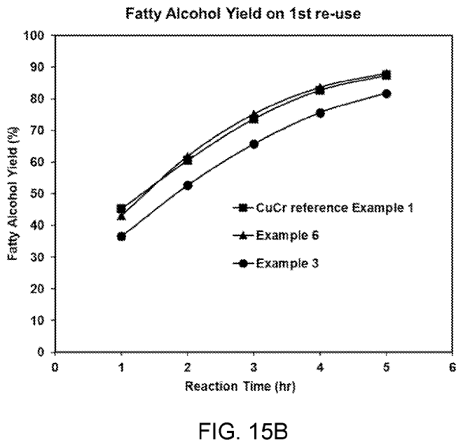

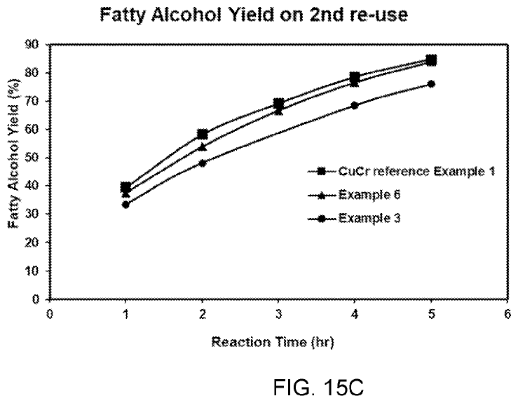

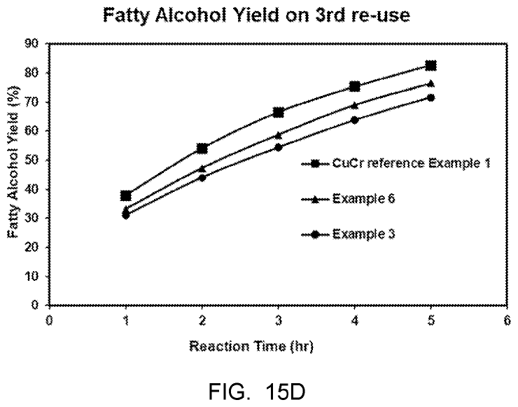

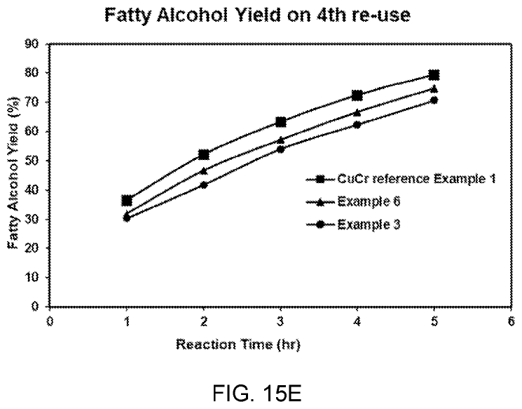

As set forth above, catalyst compositions according to embodiments herein have a higher activity than a CuCr catalyst that is currently used in commercial processes. In slurry phase process for fatty alcohol production, the catalyst stays in the system much longer than the conventional reaction time of 5 hours. Fatty alcohol yield of re-use test of CuCr reference Example 1, Example 6 (CuLa—Al₂O₃) and Example 3 (CuMn—Al₂O₃) are shown in FIGS. 15A-15E. The test conditions were the same as standard conditions.

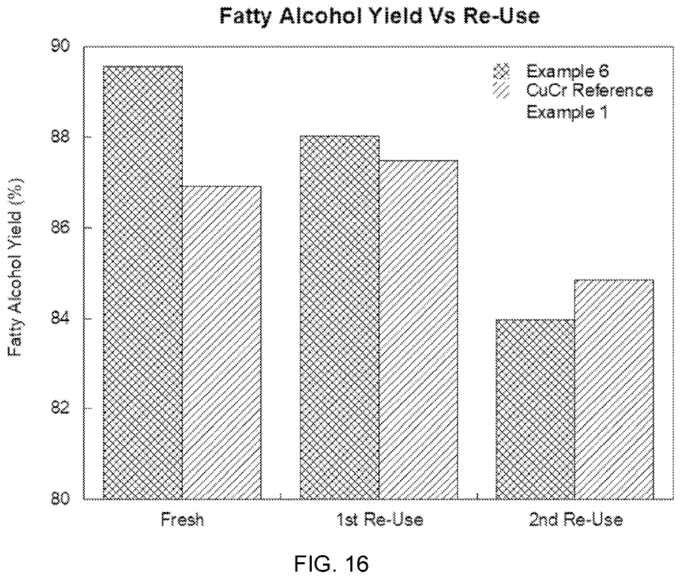

FIG. 15A a chart showing a comparison between a commercial catalyst and the catalyst compositions of Examples 3 and 6 for the first cycle (fresh) of a multi-cycle performance test. FIG. 15B a chart showing a comparison between a commercial catalyst and the catalyst compositions of Examples 3 and 6 for the second cycle (first re-use) of a multi-cycle performance test. FIG. 15C a chart showing a comparison between a commercial catalyst and the catalyst compositions of Examples 3 and 6 for the third cycle (second re-use) of a multi-cycle performance test. FIG. 15D a chart showing a comparison between a commercial catalyst and the catalyst compositions of Examples 3 and 6 for the fourth cycle (third re-use) of a multi-cycle performance test. FIG. 15E a chart showing a comparison between a commercial catalyst and the catalyst compositions of Examples 3 and 6 for the fifth cycle (fourth re-use) of a multi-cycle performance test. FIG. 16 is a chart showing a comparison between a commercial catalyst and the catalyst compositions of Example 6 for each cycle of re-use in the hydrogenolysis of methyl ester to form fatty alcohol.

As shown in FIGS. 15A-15E, each catalyst lost some activity after each use (test). This can most easily be seen by comparing the fatty alcohol yield at a constant time, which decreases for all three catalysts as the samples were reused multiple times. An equilibrium fatty alcohol yield could have been achieved during each cycle if the reaction was allowed to run long enough, but a constant time of five (5) hours was used instead for the multiple cycles. Without being bound by any particular theory, it is believed that the loss in activity from cycle to cycle can be attributed to catalyst Cu crystallite size growth and poisoning from feed impurities.

Both Example 6 and Example 3 had better fatty alcohol yield as fresh catalyst compared to the CuCr reference catalyst (Example 1). Further re-use tests showed that Example 6 (CuLa—Al₂O₃) catalyst has equal or better activity than CuCr reference catalyst (Example 1) up to 2 re-uses and then starts to show lower fatty alcohol yield due to deactivation. The Example 3 (CuMn—Al₂O₃) sample deactivated faster than the CuCr reference catalyst (Example 1) immediately upon reuse, best seen in the first reuse test results and continued to perform with lower activity in all tests thereafter.

Further analysis of the product distribution for the CuCr reference catalyst (Example 1) and the Example 6 sample showed that lower fatty alcohol yield was due to a higher concentration of fatty-fatty ester formation. This indicates that fatty-fatty ester is an intermediate in methyl ester hydrogenolysis. It was likely formed through trans-esterification of methyl ester with produced fatty alcohol. Catalysts with good activity can minimize this intermediate formation and push to complete hydrogenolysis.

### Example 18—Wax Ester Hydrogenolysis Performance Comparison

Using the procedure described below for catalyst performance comparison, the liquid product during the reaction was collected for SAP (saponification) value analysis. The SAP value is the hydrolysis of ester with KOH (or NaOH) to form alcohol and potassium or sodium salt of the corresponding acid. A higher SAP value means a higher ester content. In this case, a higher SAP value means a lower amount of ester is converted, thus, the catalyst activity would be lower.

The wax esters hydrogenolysis performance of a catalyst according to embodiments herein was compared with a commercially supplied CuCr catalyst. In these tests, hydrogenolysis product was withdrawn every hour for analysis, and subsequently new fatty acid was injected as feed in the first four hours.

After the fourth injection, as the reaction time continued, the residual wax ester reduced, and the ester conversion reduced due to approaching equilibrium. Catalysts according to embodiments herein (e.g., Example 6, calcined at 500° C.) had consistently higher wax ester conversion than the CuCr reference catalyst beginning at the second hour of testing. The overall wax ester conversion after 6 hours is summarized in Table 8. Catalyst compositions according to embodiments herein had a higher wax ester conversion than the commercial CuCr reference catalyst, 76.63% vs 72.95% under the same testing conditions. Overall, the inventive catalyst had significantly higher activity and alcohol yield.

TABLE 8
Comparison of Inventive Catalyst Activity
with Commercial CuCr Reference

| | Overall |
|---|---|
| Catalyst | Conversion, % |
| CuCr reference | 72.95 |
| (Example 1) | |
| Example 6 | 76.63 |

### Example 19—Separation and Filtration Properties

Catalyst Filtration Properties: Used catalyst separation experiments (both centrifuge separation and filtration) were conducted to compare the catalyst filterability. The results show that new CuO—Mn₂O₃—Al₂O₃ catalysts have comparable separation properties.

Procedure for Centrifugation: It's a qualitative estimate on settling and separation by centrifugation at 11,000 rpm for 5 minutes with a picture taken for visual comparison. At first, the spent catalyst slurry with the liquid products and unreacted fatty acid methyl ester (0.8 wt % catalyst loading) were centrifuged for 5 mins. Then the top 35 mL liquid was collected and a picture taken to see if any particles were floating. The separation efficiency was measured qualitatively based on the color and fine particles floating in the collected liquid. A centrifuge separation test showed that both the current inventive catalyst and CuCr had a clear liquid product.

Procedure for Filtration: 12 ml of spent catalyst slurry (well mixed) was taken in a syringe. A syringe filter (0.45 μm) with in the tip of the syringe was inserted. The syringe was pressed to have the filtrate from the syringe filter with a timer on. The filter was changed and the timer stopped if there was no filtrate coming out with a maximum hand pressure. The timer was stopped when 12 mL of the slurry was filtered through. The test qualitatively estimated the ease of filtration (by no. of filters required and time required for making same amount of filtrate) using 0.45 μm filter.

Another measurement for the success of a new catalyst was the catalyst solid-liquid product separation/filtration after reaction. Two tests were conducted for comparison. Liquid products were collected by (a) centrifuge and (b) syringe filtration. Example 6 was compared with CuCr reference catalyst (Example 1). All liquid products collected either by centrifuge or syringe filter were clear, which was a good indication of catalyst particle integrity and chemical stability after the reaction (these results were from a fresh test only).

To give a quantitative comparison of the filterability of the deposition-precipitation catalyst CuMn—Al₂O₃ Example 6 with the CuCr reference catalyst (Example 1) and the co-precipitated catalyst in Reference Example 3 CuMn—Al₂O₃, one way is to quantify the time and number of syringe filters used to collect the same amount of liquid product (12 ml). The Example 6 Catalyst (73-2) had comparable filtration time with CuCr, about 40 seconds, with CuCr reference catalyst (Example 1) and both catalysts required one syringe filter to collect 12 ml liquid product. Under the same filtration test conditions, Example 3 required 2 syringe filters to collect the same amount of liquid product and required about twice as long, 80 seconds, to collect 12 ml liquid product.

### Example 20—Spent Catalyst Particle Size Analysis After Reuses Test

The particle size distribution (PSD) of spent Reference Example 3 CuMn—Al₂O₃ and Example 6 CuLa—Al₂O₃ were similar to fresh. There was no catalyst particle size break up to a smaller size less than 0.1 μm. These results further demonstrate the stability of catalyst compositions according to embodiments herein.

### Example 21—Catalyst Particle Size Stability Test

Spent Example 7 gave an acceptable particle size distribution with no fines <0.5 μm. Example 7 had good performance and a stable PSD. This is an advantage over conventional co-precipitated CuMn—Al₂O₃ catalyst, and more importantly, has better catalytic performance than the CuCr reference catalyst (Example 1).

### Example 22—Tablet Preparation, Properties and Catalytic Performance

Powder from Example 6 was formed into a tablet by compaction. The tablet had the following properties: Packed bulk density: 1.43 g/ml; Crush strength: 30.0 lbs; BET surface area: 95.8 m2/g.

This sample was tested in a fixed bed liquid phase butyraldehyde to butanol under the following test conditions:

Reaction Conditions
Pressure: 700 psi;
Temperature: 150-180° C.;
Feed: C₄ aldehyde
LHSV: 0.75-1.5 h⁻¹
Time on stream: 150 hours
Catalyst: 30 cc
H₂/feed (mole): 5-7

This sample was tested in a fixed bed liquid phase butyraldehyde to butanol under the following test conditions:

The catalyst (30 cc) was loaded with equal volume of inerts in the reactor.

The catalyst was activated the catalyst with 3% hydrogen in nitrogen at 190° C. overnight with total gas flow rate at 1000-1500 h⁻¹ GHSV, then slowly increase the hydrogen concentration (25, 50, 75 and 100%) every 2-3 h and at 100% hydrogen concentration hold it for 1 h at 190° C. Increase the temperature to 210° C. and pass 100% hydrogen at that temperature for another hour to complete activation of the catalyst

Cool down the reactor to its reaction temperature. Pressure the reaction system to 700 psi. start the liquid flow and adjust the hydrogen to its required flow.

The liquid sample was collected every 24 hours and analyzed in GC offline.

$$n - {C}_{4} aldehyde Conversion ( \% ) = ( {nC}_{4} aldehyde in - {nC}_{4} aldehyde out ) / {nC}_{4} aldehyde in * 100$$

$$n - Butanol Selectivity ( \% ) = ( n - Butanol out - n - Butanol in ) / ( n - {C}_{4} aldehyde in - n - {C}_{4} aldehyde out ) * 100.$$

At a reaction temperature: 150° C., LHSV: 1 hr⁻¹ and time on stream: 122 hours, the butyraldehyde conversion was 99.06% and the butanol selectivity was 93.34%.

Reference throughout this specification to, for example, “one embodiment,” “certain embodiments,” “one or more embodiments” or “an embodiment” means that a particular feature, structure, material, or characteristic described in connection with the embodiment is included in at least one embodiment of the invention. Thus, the appearances of the phrases such as “in one or more embodiments,” “in certain embodiments,” “in one embodiment” or “in an embodiment” in various places throughout this specification are not necessarily referring to the same embodiment of the invention. Furthermore, the particular features, structures, materials, or characteristics may be combined in any suitable manner in one or more embodiments.

As used herein, the singular forms “a,” “an,” and “the” include plural references unless the context clearly indicates otherwise. Thus, for example, reference to “a robot arm” includes a single robot arm as well as more than one robot arm.

As used herein, the term “about” in connection with a measured quantity, refers to the normal variations in that measured quantity as expected by one of ordinary skill in the art in making the measurement and exercising a level of care commensurate with the objective of measurement and the precision of the measuring equipment. In certain embodiments, the term “about” includes the recited number ±10%, such that “about 10” would include from 9 to 11.

The term “at least about” in connection with a measured quantity refers to the normal variations in the measured quantity, as expected by one of ordinary skill in the art in making the measurement and exercising a level of care commensurate with the objective of measurement and precisions of the measuring equipment and any quantities higher than that. In certain embodiments, the term “at least about” includes the recited number minus 10% and any quantity that is higher such that “at least about 10” would include 9 and anything greater than 9. This term can also be expressed as “about 10 or more.” Similarly, the term “less than about” typically includes the recited number plus 10% and any quantity that is lower such that “less than about 10” would include 11 and anything less than 11. This term can also be expressed as “about 10 or less.”

Unless otherwise indicated, all parts and percentages are by weight. Weight percent (wt. %), if not otherwise indicated, is based on an entire composition free of any volatiles, that is, based on dry solids content.

The foregoing description discloses example embodiments of the disclosure. Modifications of the above-disclosed assemblies, apparatus, and methods which fall within the scope of the disclosure will be readily apparent to those of ordinary skill in the art. Accordingly, while the present disclosure has been disclosed in connection with example embodiments, it should be understood that other embodiments may fall within the scope of the disclosure, as defined by the following claims.

1. A catalyst composition, comprising:
a support comprising alumina;
a copper compound on the support; and
a promoter,
wherein the catalyst composition is free of chromium.

2. (canceled)

3. The catalyst composition of claim 1, wherein the alumina comprises pseudoboehmite alumina, bayerite alumina or gamma alumina.

4. (canceled)

5. (canceled)

6. The catalyst composition of claim 1, wherein the catalyst is in powder form and comprises a loose packed bulk density of about 0.25 g/mL to about 0.6 g/mL.

7. (canceled)

8. The catalyst composition of claim 1, wherein the catalyst composition is a powder comprising an average particle size of D10 of about 1 μm to about 10 μm, D50 of about 10 μm to about 20 μm, and D90 of about 30 μm to about 60 μm.

9. The catalyst composition of claim 1, having a stability against impurity poisoning of up to about 0.5% of organic acid.

10. (canceled)

11. The catalyst composition of claim 1, having a fatty acid conversion of at least about 73% based on the % saponification (SAP) value reduction.

12. (canceled)

13. The catalyst composition of claim 1, wherein the catalyst composition is calcined, and wherein a size of the catalyst composition pre-calcination is the same or about the same as the size of the catalyst composition post-calcination.

14. (canceled)

15. (canceled)

16. The catalyst composition of claim 1, wherein the copper compound is copper (II) oxide comprising a crystallite size of about 90 Å to about 200 Å.

17. The catalyst composition of claim 1, wherein the promoter comprises one or more oxide of lanthanum, manganese, barium, zirconium, calcium, magnesium, zinc, yttrium, erbium, cerium, a rare earth metal, strontium, boron, nickel, platinum, silver, gold, palladium, ruthenium or combinations thereof.

18. The catalyst composition of claim 1, wherein the copper compound is copper (II) oxide (CuO) in an amount of about 25 wt % to about 80 wt %, based on the total weight of the catalyst composition.

19. The catalyst composition of claim 1, wherein the promoter comprises barium oxide or lanthanum oxide or manganese oxide, in an amount of about 0 wt % to about 20 wt %, based on the total weight of the catalyst composition.

20. (canceled)

21. (canceled)

22. The catalyst composition of claim 1, comprising alumina in an amount of about 10 wt % to about 50 wt %, based on the total weight of the catalyst composition.

23. The catalyst composition of claim 1, wherein the copper compound comprises monoclinic copper (II) oxide (CuO), and further comprising one or more of the following crystal phases:
monoclinic lanthanum oxide carbonate (LaCO₃);
orthorhombic lanthanum copper oxide (La₂CuO₄);
tetragonal copper lanthanum oxide (CuLaO₃);
copper aluminum oxide (CuAl₄O₇);
cubic copper aluminum oxide (CuAl₂O₄);
monoclinic lanthanum manganese oxide (LaMnO_{3.13});
cubic copper manganese oxide (Cu_{1.5}Mn_{1.5}O₄);
orthorhombic lanthanum copper oxide (La₂CuO₄); or
cubic aluminum oxide (Al₂O₃).

24. The catalyst composition of claim 1, comprising a BET surface area of about 60 m²/g to about 200 m²/g.

25. The catalyst composition of claim 1, wherein the catalyst composition is in the form of at least one of a sphere, solid, tablet, caplet or slug having a thickness of about ¼ in to about 1/16 in.

26. (canceled)

27. (canceled)

28. (canceled)

29. The catalyst composition of claim 25, comprising a pore volume of about 0.25 ml/g to about 0.4 ml/g.

30. The catalyst composition of claim 25, comprising an average pore diameter of about 80 Å to about 160 Å as measured and calculated by 4 volume/area (V/A).

31. A method of preparing a chromium-free catalyst composition, comprising:
combining a copper containing solution with alumina to form a combination;
precipitating the combination with a base solution to form a precipitate solution;
filtering the precipitate solution to obtain a precipitate;
calcining the precipitate to form the catalyst composition.

32. (canceled)

33. (canceled)

34. (canceled)

35. The method of claim 31, wherein the alumina comprises pseudoboehmite alumina, bayerite alumina or gamma alumina.

36. The method of claim 31, wherein the catalyst composition is free of aluminum nitrate, an acetate, a chloride or sodium aluminate.

37. The method of claim 31, wherein the combination is a slurry, and the alumina is a powder dispersed within the slurry.

38. (canceled)

39. The method of claim 31, wherein precipitating the combination comprises combining the combination with a base.

40. (canceled)

41. (canceled)

42. (canceled)

43. (canceled)

44. (canceled)

45. The method of claim 31, wherein the catalyst composition comprises about 40 wt % to about 70 wt % copper (II) oxide (CuO) and about 30 wt % to about 60 wt % alumina.

46. (canceled)

47. (canceled)

48. (canceled)

49. A method for the hydrogenation or hydrogenolysis of organic compounds comprising carbonyl groups, the method comprising:
contacting the organic compounds with a catalyst composition according to claim 1,
wherein the catalyst composition is free of chromium.

50. The method of claim 50, wherein the organic compounds comprise one or more of aldehydes, esters, ketones or carboxylic acids.

51. The method of claim 50, wherein the organic compounds comprise one or more of fatty esters or methyl ester.

52. The method of claim 50, wherein the organic compounds comprise a methyl ester that undergoes hydrogenolysis to form one or more fatty alcohols.

53. The method of claim 50, wherein the organic compounds comprise a wax ester that undergoes hydrogenolysis to form one or more fatty alcohols.

54. The catalyst composition of claim 1, wherein the catalyst is in tablet form and comprises a bulk density of greater than about 1.0 g/mL.

55. A catalyst composition, comprising:
a support comprising gamma alumina in an amount of about 20 wt % to about 40 wt %, based on the total weight of the catalyst composition;
a copper compound on the support, wherein the copper compound is copper (II) oxide (CuO) in an amount of about 35 wt % to about 70 wt %, based on the total weight of the catalyst composition; and
a promoter, wherein the promoter comprises lanthanum oxide (La₂O₃) in an amount of about 5 wt % to about 15 wt %, based on the total weight of the catalyst composition;
wherein the catalyst composition is in the form of at least one of a sphere, solid, tablet, caplet or slug having a thickness of about ¼ in to about 1/16 in; and
wherein the catalyst composition comprises a BET SA of 20 m²/g to about 100 m²/g, and comprises a pore volume of about 0.25 mL/g to about 0.4 mL/g, and is free of chromium.

56. A method for the hydrogenation or hydrogenolysis of one or more esters, the method comprising:
contacting the organic compounds with a catalyst composition according to claim 55.
